SEQUENCE LISTING (71 sequences)

SEQ ID NO: 1 - DNA, 47 bp, synthetic construct
  source 1..47: mol_type = other DNA
tcagcattccaagaatgttctgttatgggtttatacaacaaaagcta

SEQ ID NO: 2 - DNA, 44 bp, synthetic construct
  source 1..44: mol_type = other DNA
gggacacttcgcatggtggacagtttgccaagataattacatcc

SEQ ID NO: 3 - DNA, 22 bp, synthetic construct
  source 1..22: mol_type = other DNA
tctaaagagggttttttcactt

SEQ ID NO: 4 - DNA, 19 bp, synthetic construct
  source 1..19: mol_type = other DNA
aaccatctatttgttcgcg

SEQ ID NO: 5 - DNA, 20 bp, synthetic construct
  source 1..20: mol_type = other DNA
gccacggaacctccaagagc

SEQ ID NO: 6 - DNA, 23 bp, synthetic construct
  source 1..23: mol_type = other DNA
tgtgaatgcgtcatcatctgaag

SEQ ID NO: 7 - DNA, 26 bp, synthetic construct
  source 1..26: mol_type = other DNA
gggatctcattattagtgatatgtac

SEQ ID NO: 8 - DNA, 19 bp, synthetic construct
  source 1..19: mol_type = other DNA
gtgcaactgtacatacagc

SEQ ID NO: 9 - DNA, 19 bp, synthetic construct
  source 1..19: mol_type = other DNA
tctctgatgcagattcaac

SEQ ID NO: 10 - DNA, 23 bp, synthetic construct
  source 1..23: mol_type = other DNA
aatatgtaatttgaatgcatgac

SEQ ID NO: 11 - DNA, 21 bp, synthetic construct
  source 1..21: mol_type = other DNA
ataggaagacaactgaattgg

SEQ ID NO: 12 - DNA, 21 bp, synthetic construct
  source 1..21: mol_type = other DNA
ggggaaatttactcatgtcaa

SEQ ID NO: 13 - DNA, 50 bp, synthetic construct
  source 1..50: mol_type = other DNA
accaaccttttgataatttgcaacacactatgttagaattactggcttat

SEQ ID NO: 14 - DNA, 44 bp, synthetic construct
  source 1..44: mol_type = other DNA
atgcaaaagtattctacactccaggggtagtagagagctaggcc

SEQ ID NO: 15 - DNA, 21 bp, synthetic construct
  source 1..21: mol_type = other DNA
cattaagtgcacctacactag

SEQ ID NO: 16 - DNA, 18 bp, synthetic construct
  source 1..18: mol_type = other DNA
acactatgcgagcagaag

SEQ ID NO: 17 - DNA, 24 bp, synthetic construct
  source 1..24: mol_type = other DNA
catctgagatattgagtgttgggt

SEQ ID NO: 18 - DNA, 21 bp, synthetic construct
  source 1..21: mol_type = other DNA
gaccacctggtactggtaaga

SEQ ID NO: 19 - DNA, 20 bp, synthetic construct
  source 1..20: mol_type = other DNA
gtgattattttgtgctgaca

SEQ ID NO: 20 - DNA, 20 bp, synthetic construct
  source 1..20: mol_type = other DNA
gtttaccgaggtacaacaac

SEQ ID NO: 21 - DNA, 22 bp, synthetic construct
  source 1..22: mol_type = other DNA
tctttactggttatcgtgtaac

SEQ ID NO: 22 - DNA, 24 bp, synthetic construct
  source 1..24: mol_type = other DNA
gcaggtataattctactacattta

SEQ ID NO: 23 - DNA, 20 bp, synthetic construct
  source 1..20: mol_type = other DNA
tatcaaaacactctacacga

SEQ ID NO: 24 - DNA, 17 bp, synthetic construct
  source 1..17: mol_type = other DNA
ctgtcgtctcaggcaat

SEQ ID NO: 25 - DNA, 46 bp, synthetic construct
  source 1..46: mol_type = other DNA
ttccwgcaaagacatcttcaagtctcttctatcgttccatcaggcc

SEQ ID NO: 26 - DNA, 45 bp, synthetic construct
  source 1..45: mol_type = other DNA
gaggctctcatggaatggctaaagacaaggtgagcgtgaacacaa

SEQ ID NO: 27 - DNA, 20 bp, synthetic construct
  source 1..20: mol_type = other DNA
tctaaccgaggtcgaaacgt

SEQ ID NO: 28 - DNA, 18 bp, synthetic construct
  source 1..18: mol_type = other DNA
tgcagtcctcgctcactg

SEQ ID NO: 29 - DNA, 19 bp, synthetic construct
  source 1..19: mol_type = other DNA
gcgatctcggctttgaggg

SEQ ID NO: 30 - DNA, 25 bp, synthetic construct
  source 1..25: mol_type = other DNA
caatcttgtcacctctgactaaggg

SEQ ID NO: 31 - DNA, 19 bp, synthetic construct
  source 1..19: mol_type = other DNA
gggcattttggacaaagcg

SEQ ID NO: 32 - DNA, 23 bp, synthetic construct
  source 1..23: mol_type = other DNA
agtttaactgctctrtccatgtt

SEQ ID NO: 33 - DNA, 18 bp, synthetic construct
  source 1..18: mol_type = other DNA
aactggcaagtgcaccag

SEQ ID NO: 34 - DNA, 17 bp, synthetic construct
  source 1..17: mol_type = other DNA
aggccaaaygccacttc

SEQ ID NO: 35 - DNA, 19 bp, synthetic construct
  source 1..19: mol_type = other DNA
ggrttggttgttgycacca

SEQ ID NO: 36 - DNA, 18 bp, synthetic construct
  source 1..18: mol_type = other DNA
gccatttgctccatrgcc

SEQ ID NO: 37 - DNA, 17 bp, synthetic construct
  source 1..17: mol_type = other DNA
tccatrgcctcygctgc

SEQ ID NO: 38 - DNA, 41 bp, synthetic construct
  source 1..41: mol_type = other DNA
tgagaccgatgctgtgaatcagcgctgcttttggtctagtg

SEQ ID NO: 39 - DNA, 43 bp, synthetic construct
  source 1..43: mol_type = other DNA
cagacagatggctactaccaccaacgtagtgctagccagcacc

SEQ ID NO: 40 - DNA, 18 bp, synthetic construct
  source 1..18: mol_type = other DNA
aggatgggaacagtgacc

SEQ ID NO: 41 - DNA, 20 bp, synthetic construct
  source 1..20: mol_type = other DNA
ccagccatctgttccatagc

SEQ ID NO: 42 - DNA, 21 bp, synthetic construct
  source 1..21: mol_type = other DNA
aatctgttcacaagtggcaca

SEQ ID NO: 43 - DNA, 25 bp, synthetic construct
  source 1..25: mol_type = other DNA
cactaatcaggcatgaaaacagaat

SEQ ID NO: 44 - DNA, 41 bp, synthetic construct
  source 1..41: mol_type = other DNA
tctgccagcttttggacgcagatggtctcagctatgaacac

SEQ ID NO: 45 - DNA, 42 bp, synthetic construct
  source 1..42: mol_type = other DNA
agagctgcaaagcaacattggcttccccattcttttgacttg

SEQ ID NO: 46 - DNA, 18 bp, synthetic construct
  source 1..18: mol_type = other DNA
ttcagtgcctggagtgag

SEQ ID NO: 47 - DNA, 21 bp, synthetic construct
  source 1..21: mol_type = other DNA
ccattacatcctttgcaatcc

SEQ ID NO: 48 - DNA, 21 bp, synthetic construct
  source 1..21: mol_type = other DNA
tttcccattccattcattgtt

SEQ ID NO: 49 - DNA, 18 bp, synthetic construct
  source 1..18: mol_type = other DNA
agtgctgagatctcttgg

SEQ ID NO: 50 - DNA, 15 bp, synthetic construct
  source 1..15: mol_type = other DNA
agcagagcagcgaga

SEQ ID NO: 51 - DNA, 18 bp, synthetic construct
  source 1..18: mol_type = other DNA
gcatcacattcacacagg

SEQ ID NO: 52 - DNA, 22 bp, synthetic construct
  source 1..22: mol_type = other DNA
tctgtgctttatgcgagaaaca

SEQ ID NO: 53 - DNA, 23 bp, synthetic construct
  source 1..23: mol_type = other DNA
attcaatgcaagtaaaactagga

SEQ ID NO: 54 - DNA, 19 bp, synthetic construct
  source 1..19: mol_type = other DNA
tggtcatgtacctgaatcc

SEQ ID NO: 55 - DNA, 18 bp, synthetic construct
  source 1..18: mol_type = other DNA
catggagctctgctttag

SEQ ID NO: 56 - DNA, 19 bp, synthetic construct
  source 1..19: mol_type = other DNA
tcacaagagctgaatttcc

SEQ ID NO: 57 - DNA, 23 bp, synthetic construct
  source 1..23: mol_type = other DNA
ggttcgagcattatagatatttc

SEQ ID NO: 58 - DNA, 27 bp, synthetic construct
  source 1..27: mol_type = other DNA
aaagaacaaattgaaagaatatgaaat

SEQ ID NO: 59 - DNA, 21 bp, synthetic construct
  source 1..21: mol_type = other DNA
atgaaatggagagctgataag

SEQ ID NO: 60 - DNA, 42 bp, synthetic construct
  source 1..42: mol_type = other DNA
gtccatcacgatgccagtggtatgtacgttgctatccaggct

SEQ ID NO: 61 - DNA, 38 bp, synthetic construct
  source 1..38: mol_type = other DNA
tgtgcccatctacgaggggttagtcagtcaggtcccgg

SEQ ID NO: 62 - DNA, 18 bp, synthetic construct
  source 1..18: mol_type = other DNA
gaccttcaacaccccagc

SEQ ID NO: 63 - DNA, 20 bp, synthetic construct
  source 1..20: mol_type = other DNA
cggtgaggatcttcatgagg

SEQ ID NO: 64 - DNA, 19 bp, synthetic construct
  source 1..19: mol_type = other DNA
aggcgtacagggatagcac

SEQ ID NO: 65 - DNA, 17 bp, synthetic construct
  source 1..17: mol_type = other DNA
tcccccatgccatcctg

SEQ ID NO: 66 - DNA, 500 bp, Severe acute respiratory syndrome-related coronavirus
  source 1..500: mol_type = genomic DNA
cagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaa

SEQ ID NO: 67 - DNA, 600 bp, Severe acute respiratory syndrome-related coronavirus
  source 1..600: mol_type = genomic DNA
ctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttg

SEQ ID NO: 68 - DNA, 700 bp, Influenza A virus
  source 1..700: mol_type = genomic DNA
atgagtcttctaactgaggtcgaaacgtacgttctctctatcatcccgtcaggccccctcaaagccgagatcgcacagagacttgaagatgtatttgctggaaagaataccgatcttgaggctctcatggagtggctaaagacaagaccaatcctgtcacctctgactaaggggattttaggatttgtgttcacgctcaccgtgcccagtgagcgaggactgcagcgtagacgctttgtccaaaatgcccttaatgggaatggggatccaaataatatggacagagcagtcaaactttatcgaaagcttaagagggagataacattccatggagccaaagaaatagcactcagttattctgctggtgcacttgccagttgtatgggactcatatacaacaggatgggggctgtgaccaccgaatcagcatttggccttatatgtgcaacctgtgaacagattgcagactcccagcataagtctcacaggcaaatggtaacaacaaccaatccattaataagacatgagaacagaatggttctggccagcactacagctaaggctatggagcaaatggctggatcgagcgaacaagcagctgaggccatggaggttgctagtcaggccaggcagatggtgcaggcaatgagagccattgggactcatcctagctctagcactggtctgaaaaatgatctcc

SEQ ID NO: 69 - DNA, 420 bp, Influenza A virus
  source 1..420: mol_type = genomic DNA
tccaaataacatggacaaagcagttaaactgtataggaaacttaagagggagataacgttccatggggccaaagaaatagctctcagctattctgctggtgcacttgccagttgcatgggcctcatatacaataggatgggggctgtaaccactgaagtggcatttggcctggtatgtgcaacatgtgagcagattgctgactcccagcacaggtctcataggcaaatggtggcaacaaccaatccattaataagacatgagaacagaatggtcttggccagcactacagctaaggctatggagcaaatggctggatcaagtgagcaggcagcggaggccatggagattgctagtcaggccaggcagatggtgcaggcaatgagagccattgggactcatcctagttccagtactggtct

SEQ ID NO: 70 - DNA, 560 bp, Influenza B virus
  source 1..560: mol_type = genomic DNA
agggcctgattctggctgagagaaaaatgagaaaatgtgtgagctttcatgaagcatttgaaatagcagaaggccatgaaagctcagcgctactatattgtctcatggtcatgtacctgaatcctggaaattattcaatgcaagtaaaactaggaacactctgtgctttgtgcgaaaaacaagcatcacattcacacagagctcatagcagagcagcgagatcctcagtgccaggagtgagacgggaaatgcagatggtctcagctatgaacacagcaaaaacaatgaatggaatgggaaaaggagaagacgtccaaaaactggcagaagaactgcaaagcaacattggagtattgagatcccttggggcaagtcagaagaatggagaaggaattgcaaaagatgtaatggaagtgctaaagcagagctccatgggaaattcagctcttgtgaagaaatacctataatgtttgaaccattccagattctttcaatttgttcttttattctatcagctctccatttcatggcttggacaataggacatttaaatcaaataa

SEQ ID NO: 71 - DNA, 280 bp, Homo sapiens
  source 1..280: mol_type = genomic DNA
aaggccaaccgcgagaagatgacccagatcatgtttgagaccttcaacaccccagccatgtacgttgctatccaggctgtgctatccctgtacgcctctggccgtaccactggcatcgtgatggactccggtgacggggtcacccacactgtgcccatctacgaggggtatgccctcccccatgccatcctgcgtctggacctggctggccgggacctgactgactacctcatgaagatcctcaccgagcgcggctacagcttcaccaccacggccgagc

(19) United States
(12) Patent Application Publication
(10) Pub. No.: US 2026/0085367 A1
(43) Pub. Date: Mar. 26, 2026

(54) LAMP ASSAY SYSTEM FOR DETECTION OF RESPIRATORY VIRUS

(71) Applicant: Delta Electronics Int’l (Singapore) Pte Ltd, Singapore (SG)

(72) Inventors: Zee Hong Goh, Singapore (SG); Weishi Zhang, Singapore (SG); Jia Hui Esther Boey, Singapore (SG)

(21) Appl. No.: 18/898,476

(22) Filed: Sep. 26, 2024

(51) Int. Cl. C12Q 1/70

(52) U.S. Cl. CPC C12Q 1/701; C12Q 2600/16

(57) ABSTRACT

A LAMP assay system for detection of a respiratory virus is disclosed. The LAMP system includes a LAMP reaction mixture having a primer kit, a strand-displacing polymerase and deoxyribonucleoside triphosphates for amplifying a target sequence. The primer kit includes at least one of a first primer set specific to SARS-CoV-2, a second primer set specific to Influenza A, and a third primer set specific to Influenza B. The first primer set includes primers having nucleotide sequences of SEQ ID NOs: 1 to 6 and SEQ ID NOs: 13 to 18. The second primer set includes primers having nucleotide sequences of SEQ ID NOs: 25 to 30. The third primer set includes primers having nucleotide sequences of SEQ ID NOs: 44 to 49.

### FIELD OF THE INVENTION

The present disclosure relates to a loop-mediated isothermal amplification (LAMP) assay system, and more particularly to a LAMP assay system for detection of a respiratory virus.

### INCORPORATION BY REFERENCE

The Sequence Listing XML having the file name “08677PUS.xml”, a production date of Sep. 20, 2024, and a file size of 65 KB is incorporated herein by reference for all purposes.

### BACKGROUND OF THE INVENTION

Coronavirus disease 2019 (COVID-19) or severe acute respiratory syndrome coronavirus 2 (SARS-CoV-2) is a newly emerged betacoronaviruses that causes pneumonia and other complications. High mutation rate in viral replication for RNA viruses lead to the development of variant strains for SARS-CoV-2 which shows to be more virulent and higher in spreading rate. As herd immunity and community vaccination become the goal of countries in dealing this pandemic, some preliminary evidence suggests that fully vaccinated people will also be infected by the variant and spread the virus to others who are not eligible for vaccination.

Influenza A virus (FluA) is a negative-sense, single-stranded, segmented RNA viruses belongs to the family Orthomyxoviridae under the genus Alphainfluenzavirus. There are several subtypes for Influenza A virus based on the type of hemagglutinin and neuraminidase. Influenza A is a contagious viral infection that attacks the respiratory of the patient. Influenza A is commonly found in avian species and human. Many sub-types of Influenza A was identified depending the combination of the hemagglutinin and neuraminidase genes suggesting the complexity and diversity of the influenza A virus.

Influenza B virus (FluB) is a negative-sense, single-stranded, segmented RNA viruses belongs to the family Orthomyxoviridae under the genus Betainfluenzavirus. Different from Influenza A virus, the virus is not divided into subtypes but lineage. Influenza B viruses has a slower mutation rate in terms of their genetic and antigenic properties than influenza A viruses. As compared with Influenza A, Influenza B is more commonly found in human and seals. As compared to Influenza A, Influenza B is classified as lineage. Currently 2 major lineages were identified namely B(Victoria) and B(Yamagata).

Although seasonal flu vaccines were given to individuals to protect against flu infection but these Quadrivalent Influenza Vaccine protect against H1N1, H3N2, B(Victoria) and B(Yamagata) suggesting the risk of other FluA sub-type infection. Besides, Quadrivalent Influenza Vaccine might not be under the government funded program suggesting individuals required to vaccinate at their own expenses.

Based on the World Health Organization (WHO) estimation, a total of 290,000 to 650,000 people die of flu-related causes every year worldwide. The common symptom showed similarity with SARS-CoV-2 infection suggesting it is easily misinterpreted with among each other without further analysis. Both SARS-CoV-2 and Flu come with different treatment method. Nucleic acid tests (NAT) will be the most accurate tool to identify the current virus even strain and provide the most suitable treatment.

As many countries are achieving high community vaccination rate for SARS-CoV-2, the number of severe illness, hospitalization and death were significantly reduced suggesting the effectiveness of developed vaccines. In order to reduce the economic burden caused by COVID-19 in countries, many countries are moving forward to treat COVID-19 as pandemic to endemic. As the vaccination rate increased globally, asymptomatic or minor symptomatic patients showed the capability of self-immune system to neutralize the viruses, so the future trend of handling of COVID19 cases will be facing strategical shifting. On the other hand, the symptoms caused by Influenza A, B and COVID19 viruses are similar. Therefore, to different these respiratory viruses become a pressing need in the near future to provide evidence for proper clinical treatment and public health surveillance and management.

Disease diagnosis can be commonly classified as disease antigen detection, disease nucleic acid detection or even antibody detection. Using COVID19 as an example, there are currently two primary types of tests being used: molecular tests (also known as nucleic acid tests (NAT)) and rapid antigen tests (ART). The NAT which employed qPCR or qRT-PCR technologies served as a gold standard in some of the countries as it is more accurate than the ART. However, the trade-off is that most traditional NAT results come back in around 24 hours, and some may take longer depending on if they're sent to an outside laboratory. While for the ART, they can give results in as little as 15 minutes and have proven to be much cheaper to produce. The problem, though, is that the ART is not as sensitive as the NAT (˜1000 time less sensitive), so more likely to receive false negative and false positive results suggesting low specificity, sensitivity and the reliability of the ART test when comparing with the NAT.

Although the ART was used as home-testing devices in many countries, many drawbacks halt this initiative especially in the countries with lower vaccination rate. As aforementioned, the reason behind might be the sensitivity of the ART which detect the viral antigen showed less promising in detecting early infection as qRT-PCR. Long turnaround time in the traditional NAT reduced the effectiveness in early diagnostic and protection measurement in pathogen transmission. Additionally, the traditional NAT is bulky, costly and sophisticated, and thus is unfavourable for lay person usage and home based setting.

Therefore, in order to differentiate from the increasing crowded market of POC molecular diagnostics, it is necessary to develop novel products that have a shorter turnaround time with equivalent sensitivity and specificity as qPCR but as fast as the ART. This will ensure the patients are treated as soon as possible, and to limit the spread of diseases. Therefore after, another type of NAT with short turnaround time and lower capital investment known as Loop mediated isothermal amplification (LAMP) was developed in 2000. LAMP showed its capability in amplifying nucleic acid within short duration in single temperature has been widely developed and employed in diagnostic sector. The initial design of LAMP generating precipitation during the process allows the user to detect the amplification status via a turbidimeter. Improved version LAMP incorporated the double stranded DNA intercalating dye allows the user to observe real-time amplification of LAMP via a fluorescence detector. Both of the prior art required significant capital investment. While the invention of colorimetric LAMP in 2018 by NEB allows the user to understand the target status via color change which can be differentiable via naked eye. This further reduced the costing of LAMP amplification and allows the penetration to the rural area with less technology investment.

Although colorimetric LAMP provide diagnostic solution to rural areas but the major drawback which halt it to be sold as over the counter (OTC) kit will be the temperature stability. Reagent master mixture required cold-chain for delivery and storage increase the overall costing of the diagnostic assay.

Therefore, there is a need of providing an assay system to address the challenges encountered in the prior arts.

### SUMMARY OF THE INVENTION

An object of the present disclosure is to provide a LAMP assay system for fast detection of SARS-CoV-2, FluA and FluB.

Another object of the present disclosure is to provide a colorimetric LAMP reaction mixture which allows the user to lyophilize the whole mixture to achieve temperature stability.

An additional object of the present disclosure is to provide an extraction-free lysis buffer which is compatible to the colorimetric LAMP reaction mixture.

In accordance with an aspect of the present disclosure, a LAMP assay system for detection of a respiratory virus is provided. The LAMP system includes a LAMP reaction mixture having a primer kit, a strand-displacing polymerase and deoxyribonucleoside triphosphates for amplifying a target sequence. The primer kit includes at least one of a first primer set specific to SARS-CoV-2, a second primer set specific to Influenza A, and a third primer set specific to Influenza B. The first primer set includes primers having nucleotide sequences of SEQ ID NOs: 1 to 6 and SEQ ID NOs: 13 to 18. The second primer set includes primers having nucleotide sequences of SEQ ID NOs: 25 to 30. The third primer set includes primers having nucleotide sequences of SEQ ID NOs: 44 to 49.

In an embodiment, the first primer set further includes at least one of primers having nucleotide sequences of SEQ ID NOs: 7 to 12.

In an embodiment, the first primer set further includes at least one of primers having nucleotide sequences of SEQ ID NOs: 19 to 24.

In an embodiment, the second primer set further includes at least one of primers having nucleotide sequences of SEQ ID NOs: 31 to 37.

In an embodiment, the second primer set further includes at least one of primers having nucleotide sequences of SEQ ID NOs: 38 to 43.

In an embodiment, the third primer set further includes at least one of primers having nucleotide sequences of SEQ ID NOs: 50 to 59.

In an embodiment, the primer kit further includes an internal control primer set specific to β-actin, and the internal control primer set includes primers having nucleotide sequences of SEQ ID NOs: 60 to 65.

In an embodiment, the LAMP reaction mixture further includes a pH indicating dye in a concentration ranged 0.08 to 0.3 mM.

In an embodiment, the pH indicating dye is phenol red or neutral red.

In an embodiment, the LAMP reaction mixture further includes a lyoprotectant sugar in a concentration ranged 1 to 10% (w/v), wherein the lyoprotectant sugar is selected from the group consisting of trehalose, raffinose, dextran, mannitol and mixtures thereof.

In an embodiment, the LAMP reaction mixture includes 2 to 7.5% (w/v) of trehalose and 2 to 7.5% (w/v) of raffinose.

In an embodiment, the LAMP reaction mixture includes 2 to 7.5% (w/v) of trehalose, 2 to 7.5% (w/v) of raffinose, 1 to 2.5% (w/v) of dextran, and 1 to 5% (w/v) of mannitol.

In an embodiment, the LAMP reaction mixture further includes a magnesium salt in a concentration ranged 4 to 12 mM.

In an embodiment, the LAMP assay system further includes an extraction-free lysis buffer. The extraction-free lysis buffer includes potassium chloride in a concentration ranged 10 to 50 mM; ammonium sulfate in a concentration ranged 10 to 50 mM; and a detergent in a concentration ranged 0.5 to 6% (w/v), wherein the detergent is selected from the group consisting of 2-ethylhexan-1-ol;2-methyloxirane;oxirane (CAS number: 64366-70-7), secondary alcohol ethoxylate (CAS number: 84133-50-6), 2-[4-(2,4,4-trimethylpentan-2-yl)phenoxy]ethanol (CAS number: 9002-93-1), 2-[4-(2,4,4-trimethylpentan-2-yl)phenoxy]ethan-1-ol (CAS number: 9002-93-1), and {2-[3,4-bis(2-hydroxyethoxy)oxolan-2-yl]-2-(2-hydroxyethoxy)ethoxy}ethyl dodecanoate (CAS number: 9005-64-5) and mixtures thereof. The lyophilized colorimetric LAMP reaction mixture is rehydrated with the extraction-free lysis buffer to be ready for nucleic acid amplification and detection.

In an embodiment, the LAMP reaction mixture is all-in-one lyophilized mixture and is rehydrated with the extraction-free lysis buffer to be ready for nucleic acid amplification and detection.

In an embodiment, the detergent in the extraction-free lysis buffer is 0.5 to 6% (w/v) of 2-ethylhexan-1-ol;2-methyloxirane;oxirane (CAS number: 64366-70-7).

In an embodiment, the detergent in the extraction-free lysis buffer is 0.5 to 6% (w/v) of secondary alcohol ethoxylate (CAS number: 84133-50-6).

In an embodiment, the detergent in the extraction-free lysis buffer is 0.5 to 6% (w/v) of 2-[4-(2,4,4-trimethylpentan-2-yl)phenoxy]ethanol (CAS number: 9002-93-1).

In an embodiment, the detergent in the extraction-free lysis buffer is 0.5 to 6% (w/v) of 2-[4-(2,4,4-trimethylpentan-2-yl)phenoxy]ethan-1-ol (CAS number: 9002-93-1).

In an embodiment, the detergent in the extraction-free lysis buffer is 0.5 to 6% (w/v) of {2-[3,4-bis(2-hydroxyethoxy)oxolan-2-yl]-2-(2-hydroxyethoxy)ethoxy}ethyl dodecanoate (CAS number: 9005-64-5).

The above contents of the present disclosure will become more readily apparent to those ordinarily skilled in the art after reviewing the following detailed description and accompanying drawings, in which:

### BRIEF DESCRIPTION OF THE DRAWINGS

FIG. 1 shows partial cDNA sequence of SARS-CoV-2 and annealing sites of the primers targeting the 2-ORMT gene;

FIG. 2 shows partial cDNA sequence of SARS-CoV-2 and annealing sites of the primers targeting the Helicase gene;

FIG. 3 shows partial cDNA sequence of FluA and annealing sites of the primers targeting the M gene of FluA;

FIG. 4 shows partial cDNA sequence of FluA and annealing sites of the primers targeting the M gene of FluA;

FIG. 5 shows partial cDNA sequence of FluB and annealing sites of the primers targeting the M gene of FluB;

FIG. 6 shows partial cDNA sequence of β-actin and annealing sites of the primers targeting the β-actin gene;

FIG. 7A shows the comparison of the fluorescence LAMP with the colorimetric LAMP using SARS-CoV-2 reference RNA, FIG. 7B shows the comparison of the fluorescence LAMP with the colorimetric LAMP using FluA H1N1 reference RNA, and FIG. 7C shows the comparison of the fluorescence LAMP with the colorimetric LAMP using FluB reference RNA;

FIG. 8A shows the summary detection rate of different sample inputs for SARS-CoV-2 assay, while FIG. 8B and FIG. 8C show the amplification results in color change and fluorescence signal, respectively, of different sample inputs for SARS-CoV-2 assay;

FIG. 9A shows the summary detection rate of different sample inputs for FluA assay, while FIG. 9B and FIG. 9C show the amplification results in color change and fluorescence signal, respectively, of different sample inputs for FluA assay;

FIG. 10A shows the summary detection rate of different sample inputs for FluB assay, while FIG. 10B and FIG. 10C show the amplification results in color change and fluorescence signal, respectively, of different sample inputs for FluB assay;

FIG. 11A and FIG. 11B show the color change and the fluorescence signal detected by SARS-CoV-2 assay while using various SARS-CoV-2 strains;

FIG. 12A and FIG. 12B show the specificity analysis of SARS-CoV-2 assay with different respiratory pathogens;

FIG. 13A and FIG. 13B show the color change and the fluorescence signal detected by FluA assay while using various FluA H1 and H3 strains;

FIG. 14A and FIG. 14B show the specificity analysis of FluA assay with different respiratory pathogens;

FIG. 15A and FIG. 15B show the color change and the fluorescence signal detected by FluB assay while using various FluB strains;

FIG. 16A and FIG. 16B show the specificity analysis of FluB assay with different respiratory pathogens;

FIG. 17 shows the color change and the fluorescence signal detected by β-actin assay with different donor samples;

FIG. 18A and FIG. 18B show the multiple detection of SARS-CoV-2 and β-actin directly from the nasal sample without purification;

FIG. 19 shows the final working concentrations of the primers for the assays used in the LAMP reaction;

FIGS. 20A to 20C show the Cq values detected using 10× and 3× LoD of FluA H1N1 reference materials with the lyophilized beads stored in different temperature across different durations; and

FIGS. 20D and 20E show the color change and the Cq values detected using 10× and 3× LoD of FluA H1N1 reference materials with the lyophilized beads stored in room temperature.

### DETAILED DESCRIPTION OF THE PREFERRED EMBODIMENT

The present disclosure will now be described more specifically with reference to the following embodiments. It is to be noted that the following descriptions of the embodiments of this disclosure are presented herein for purpose of illustration and description only; it is not intended to be exhaustive or to be limited to the precise form disclosed.

The present disclosure provides a loop-mediated isothermal amplification (LAMP) assay system for detecting respiratory viruses including severe acute respiratory syndrome coronavirus 2 (SARS-CoV-2), Influenza A (FluA) and Influenza B (FluB). The LAMP assay system includes a LAMP reaction mixture having a LAMP-based primer kit, a strand-displacing polymerase and deoxyribonucleoside triphosphates (dNTPs) for amplifying a target sequence. Particularly, the LAMP-based primer kit is designed to detect SARS-CoV-2, FluA and FluB with high sensitivity and specificity. Moreover, the LAMP-based primer kit includes additional outer primers designed for SARS-CoV-2, FluA and FluB to further improve the assay detection limit.

Preferably, the LAMP assay system utilizes a modified LAMP with a pH indicator, also known as colorimetric LAMP, for rapid, sensitive and specific diagnosis of viral genomic materials via naked eyes. That is, the LAMP reaction mixture includes a pH indicating dye capable of providing color change in visual light to allow the user to differentiate the amplification results. Alternatively, the LAMP assay system is also compatible with a fluorescence LAMP reagent, which includes an intercalating fluorescence dye to detect the amplification results via fluorescence detector. The LAMP assay system is capable of amplifying the target nucleic acid in less than 20 minutes with high sensitivity and the signal can be either observed with naked eye or detected by optical systems for colorimetric or fluorescence signals.

Preferably, the LAMP reaction mixture further includes a lyoprotectant sugar, which is able to protect the LAMP reaction mixture especially the enzyme from degradation during the freeze-drying process. By including the optimal lyoprotectant sugar, the LAMP reaction mixture of the present disclosure is able to be all-in-one lyophilized into a lyophilized ready-to-use reagent after the freeze-drying process.

The LAMP assay system of the present disclosure can detect nucleic acids from standard extraction procedure or from direct extraction protocol without purification. Preferably, the LAMP assay system includes an extraction-free lysis buffer, also called direct lysis buffer, which is compatible with the LAMP reaction mixture. The direct lysis buffer includes special selections of a chaotropic salt and a detergent for ideal extraction of nucleic acids. The direct lysis buffer is capable of lysis and direct nucleic acid extraction of viruses, such as SARS-CoV-2, FluA and FluB, but not limited thereto. The lyophilized LAMP reaction mixture is able to be rehydrated with the direct lysis buffer and ready for nucleic acid amplification and detection.

The primer kit of the LAMP reaction mixture includes at least one of a first primer set specific to SARS-CoV-2, a second primer set specific to FluA, and a third primer set specific to FluB. The three primer sets may be independently used for detecting SARS-CoV-2, FluA and FluB respectively, and may also be combined to be a multiplex detection kit for detecting SARS-CoV-2, FluA and FluB simultaneously. The designs of the primers in the three primer sets are described in detail as follows.

Basically, the LAMP primer set include four primers targeting six regions of nucleic acid strands, named F3, F2, F1C, B1C, B2, and B3 (C means complementary, and F1C may also be expressed as F1′). The six regions F3, F2, F1, B1C, B2C and B3C are located in order from 5′ end to 3′ end of the forward strand in double stranded DNA, meaning the region F3 is upstream of the region F2 which in turn upstream of the region F1 and so on. Therefore, the regions F3 and B3 are located at the most outside while the regions F1 and B1C are at the most inside. Oligonucleotides targeting the regions F1C and F2 are connected tail-to-head with or without additional nucleotide to form one primer named FIP (Forward Internal Primer). Oligonucleotides targeting the regions B1C and B2 are connected tail-to-head with or without additional nucleotide to form one primer named BIP (Backward Internal Primer). Further inclusion of loop primers LF (Loop Forward) and LB (Loop Backward) targeting regions between F2C and F1C and between B1C and B2C can accelerate reaction speed. In such cases, there are 6 LAMP primers targeting 8 regions of nucleic acid strands. The primers are substantially complementary to their targeting regions for stable annealing and extension under conditions provided. Functionally, the primers F3 and B3 act as displacement primers, causing displacement and release of downstream polynucleotides generated by extension of the primers FIP and BIP.

Except the original six primers (i.e. FIP, BIP, F3, B3, LF and LB), the primer set of the present disclosure further includes additional outer primers designed to target sequences located outside the region from F3 to B3 of the original LAMP primer design. The additional outer primers are designed to increase detection sensitivity of LAMP-based nucleic acid amplification. The additional outer primers target regions outside the regions F3 and B3C, and can be introduced singly, in pairs and combination of both. The additional outer primers are located upstream of the primers F3 and B3 and have the same orientation as the primers F3 and B3. They are functioned as displacement primers as the primers F3 and B3.

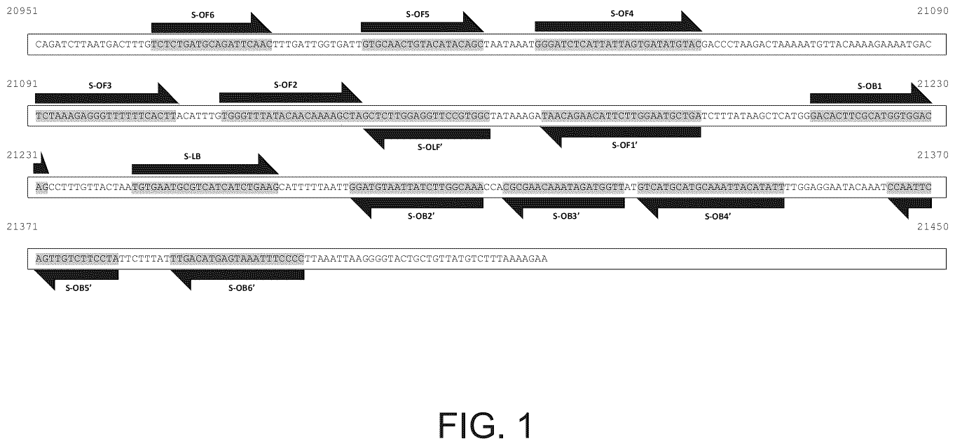

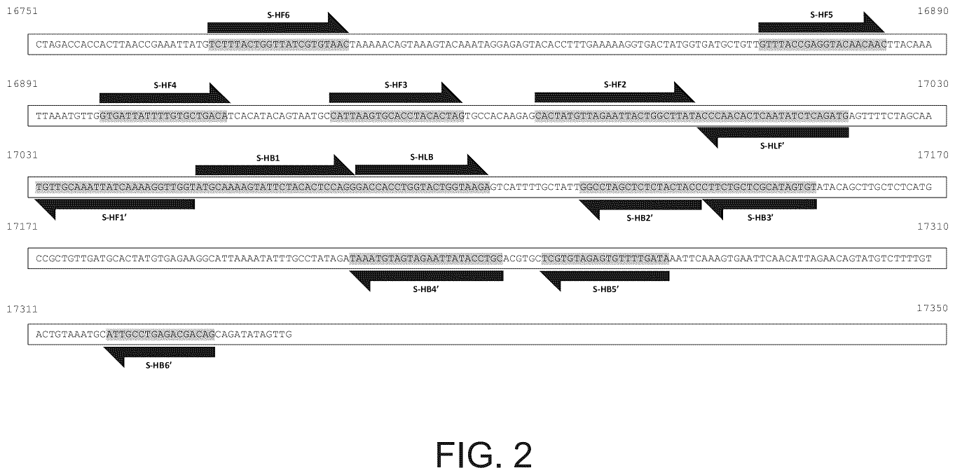

For detecting SARS-CoV-2, the first primer set includes multiple LAMP primers targeting the 2′-O-ribose methyltransferase (2-ORMT) gene and the Helicase gene located on the ORF1ab of SARS-CoV-2. The method for detecting SARS-CoV-2 is based on the reverse transcription loop-mediated isothermal amplification (RT-LAMP) technology, including the reverse transcription of converting the viral genomic RNA from the virus to a complementary DNA (cDNA) and then the isothermal amplification whereby the cDNA is amplified with the first primer set. Table 1 lists the first primer set including SEQ ID NO: 1 to SEQ ID NO: 24 for detecting SARS-CoV-2. FIG. 1 shows partial cDNA sequence of SARS-CoV-2 (SEQ ID NO: 66, which is from position 20951 to position 21450 of NCBI Reference Sequence: NC_045512.2) and annealing sites of the primers targeting the 2-ORMT gene. FIG. 2 shows partial cDNA sequence of SARS-CoV-2 (SEQ ID NO: 67, which is from position 16751 to position 17350 of NCBI Reference Sequence: NC_045512.2) and annealing sites of the primers targeting the Helicase gene. In some embodiments, a few nucleotides of the primers in Table 1 are adjusted (e.g. point mutations) to be applied for different SARS-CoV-2 variants and thus are not completely complementary to the NC_045512.2 sequence.

TABLE 1

| | | | SEQ |
|---|---|---|---|
| | Primer | | ID |
| Target | Name | Primer Sequence (5′ to 3′) | NO. |
| SARS- | S-OFIP | TCAGCATTCCAAGAATGTTCTGTTATGGGTTTATACAACAAAAGCTA | 1 |
| CoV-2 | S-OBIP | GGGACACTTCGCATGGTGGACAGTTTGCCAAGATAATTACATCC | 2 |
| | S-OF3 | TCTAAAGAGGGTTTTTTCACTT | 3 |
| | S-OB3 | AACCATCTATTTGTTCGCG | 4 |
| | S-OLF | GCCACGGAACCTCCAAGAGC | 5 |
| | S-OLB | TGTGAATGCGTCATCATCTGAAG | 6 |
| | S-OF4 | GGGATCTCATTATTAGTGATATGTAC | 7 |
| | S-OF5 | GTGCAACTGTACATACAGC | 8 |
| | S-OF6 | TCTCTGATGCAGATTCAAC | 9 |
| | S-OB4 | AATATGTAATTTGAATGCATGAC | 10 |
| | S-OB5 | ATAGGAAGACAACTGAATTGG | 11 |
| | S-OB6 | GGGGAAATTTACTCATGTCAA | 12 |
| | S-HFIP | ACCAACCTTTTGATAATTTGCAACACACTATGTTAGAATTACTGGCTTAT | 13 |
| | S-HBIP | ATGCAAAAGTATTCTACACTCCAGGGGTAGTAGAGAGCTAGGCC | 14 |
| | S-HF3 | CATTAAGTGCACCTACACTAG | 15 |
| | S-HB3 | ACACTATGCGAGCAGAAG | 16 |
| | S-HLF | CATCTGAGATATTGAGTGTTGGGT | 17 |
| | S-HLB | GACCACCTGGTACTGGTAAGA | 18 |
| | S-HF4 | GTGATTATTTTGTGCTGACA | 19 |
| | S-HF5 | GTTTACCGAGGTACAACAAC | 20 |
| | S-HF6 | TCTTTACTGGTTATCGTGTAAC | 21 |
| | S-HB4 | GCAGGTATAATTCTACTACATTTA | 22 |
| | S-HB5 | TATCAAAACACTCTACACGA | 23 |
| | S-HB6 | CTGTCGTCTCAGGCAAT | 24 |

The first primer set for detecting SARS-CoV-2 basically includes S-OFIP (SEQ ID NO: 1), S-OBIP (SEQ ID NO: 2), S-OF3 (SEQ ID NO: 3), S-OB3 (SEQ ID NO: 4), S-OLF (SEQ ID NO: 5) and S-OLB (SEQ ID NO: 6) targeting the 2-ORMT gene, and S-HFIP (SEQ ID NO: 13), S-HBIP (SEQ ID NO: 14), S-HF3 (SEQ ID NO: 15), S-HB3 (SEQ ID NO: 16), S-HLF (SEQ ID NO: 17) and S-HLB (SEQ ID NO: 18) targeting the Helicase gene. In an embodiment, the first primer set further includes additional outer primers S-OF4 (SEQ ID NO: 7), S-OF5 (SEQ ID NO: 8), S-OF6 (SEQ ID NO: 9), S-OB4 (SEQ ID NO: 10), S-OB5 (SEQ ID NO: 11) and S-OB6 (SEQ ID NO: 12) targeting the 2-ORMT gene. In an embodiment, the first primer set further includes additional outer primers S-HF4 (SEQ ID NO: 19), S-HF5 (SEQ ID NO: 20), S-HF6 (SEQ ID NO: 21), S-HB4 (SEQ ID NO: 22), S-HB5 (SEQ ID NO: 23) and S-HB6 (SEQ ID NO: 24) targeting the Helicase gene.

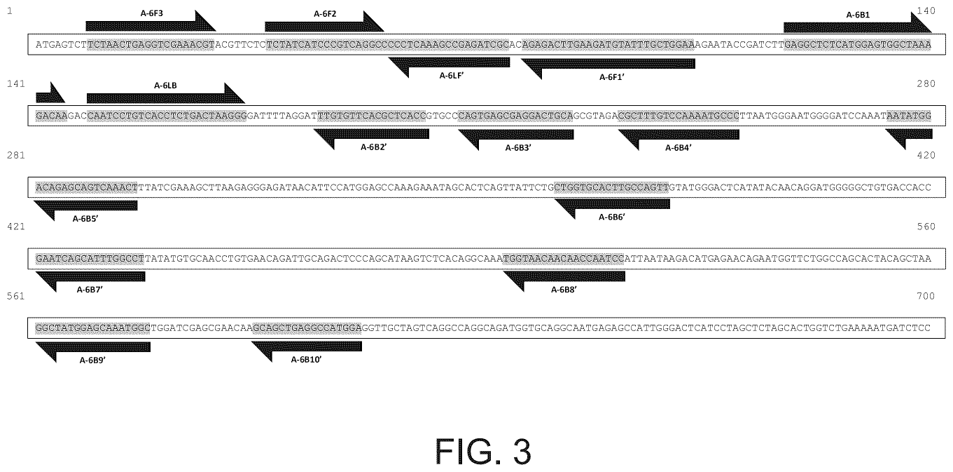

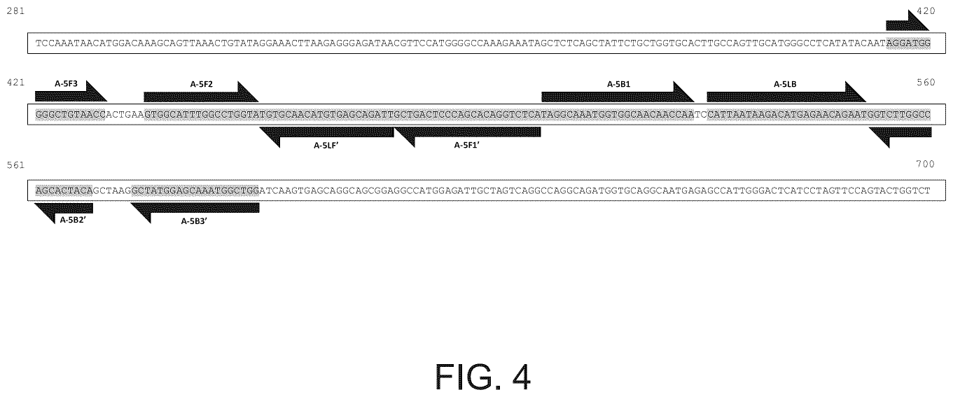

For detecting FluA, the second primer set includes multiple LAMP primers targeting the Matrix Protein (M) gene of FluA. The method for detecting FluA is based on the RT-LAMP technology, including the reverse transcription of converting the viral genomic RNA from the virus to a complementary DNA (cDNA) and then the isothermal amplification whereby the cDNA is amplified with the second primer set. Table 2 lists the second primer set including SEQ ID NO: 25 to SEQ ID NO: 43 for detecting FluA. FIG. 3 shows partial cDNA sequence of FluA (SEQ ID NO: 68, which is from position 1 to position 700 of GenBank: KC780092.1) and annealing sites of the primers targeting the M gene of FluA. FIG. 4 shows partial cDNA sequence of FluA (SEQ ID NO: 69, which is from position 281 to position 700 of GenBank: KJ609217.1) and annealing sites of the primers targeting the M gene of FluA. In some embodiments, a few nucleotides of the primers in Table 2 are adjusted (e.g. point mutations) to be applied for different FluA variants and thus are not completely complementary to the KC780092.1 sequence or the KJ609217.1 sequence.

TABLE 2

| | | | SEQ |
|---|---|---|---|
| | Primer | | ID |
| Target | Name | Primer Sequence (5′ to 3′) | NO. |
| FluA | A-6FIP | TTCCWGCAAAGACATCTTCAAGTCTCTTCTATCGTTCCATCAGGCC | 25 |
| | A-6BIP | GAGGCTCTCATGGAATGGCTAAAGACAAGGTGAGCGTGAACACAA | 26 |
| | A-6F3 | TCTAACCGAGGTCGAAACGT | 27 |
| | A-6B3 | TGCAGTCCTCGCTCACTG | 28 |
| | A-6LF | GCGATCTCGGCTTTGAGGG | 29 |
| | A-6LB | CAATCTTGTCACCTCTGACTAAGGG | 30 |
| | A-6B4 | GGGCATTTTGGACAAAGCG | 31 |
| | A-6B5 | AGTTTAACTGCTCTRTCCATGTT | 32 |
| | A-6B6 | AACTGGCAAGTGCACCAG | 33 |
| | A-6B7 | AGGCCAAAYGCCACTTC | 34 |
| | A-6B8 | GGRTTGGTTGTTGYCACCA | 35 |
| | A-6B9 | GCCATTTGCTCCATRGCC | 36 |
| | A-6B10 | TCCATRGCCTCYGCTGC | 37 |
| | A-5FIP | TGAGACCGATGCTGTGAATCAGCGCTGCTTTTGGTCTAGTG | 38 |
| | A-5BIP | CAGACAGATGGCTACTACCACCAACGTAGTGCTAGCCAGCACC | 39 |
| | A-5F3 | AGGATGGGAACAGTGACC | 40 |
| | A-5B3 | CCAGCCATCTGTTCCATAGC | 41 |
| | A-5LF | AATCTGTTCACAAGTGGCACA | 42 |
| | A-5LB | CACTAATCAGGCATGAAAACAGAAT | 43 |

The second primer set for detecting FluA basically includes A-6FIP (SEQ ID NO: 25), A-6BIP (SEQ ID NO: 26), A-6F3 (SEQ ID NO: 27), A-6B3 (SEQ ID NO: 28), A-6LF (SEQ ID NO: 29) and A-6LB (SEQ ID NO: 30) targeting the M gene of FluA. In an embodiment, the second primer set further includes additional outer primers A-6B4 (SEQ ID NO: 31), A-6B5 (SEQ ID NO: 32), A-6B6 (SEQ ID NO: 33), A-6B7 (SEQ ID NO: 34), A-6B8 (SEQ ID NO: 35), A-6B9 (SEQ ID NO: 36) and A-6B10 (SEQ ID NO: 37) targeting the M gene of FluA. In an embodiment, the second primer set further includes additional LAMP primers A-5FIP (SEQ ID NO: 38), A-5BIP (SEQ ID NO: 39), A-5F3 (SEQ ID NO: 40), A-5B3 (SEQ ID NO: 41), A-5LF (SEQ ID NO: 42) and A-5LB (SEQ ID NO: 43) targeting the M gene of FluA.

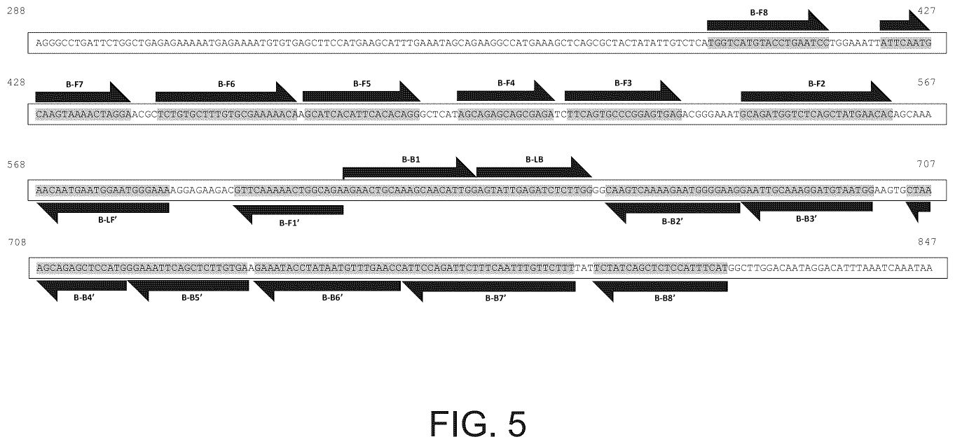

For detecting FluB, the third primer set includes multiple LAMP primers targeting the Matrix Protein (M) gene of FluB. The method for detecting FluB is based on the RT-LAMP technology, including the reverse transcription of converting the viral genomic RNA from the virus to a complementary DNA (cDNA) and then the isothermal amplification whereby the cDNA is amplified with the third primer set. Table 3 lists the third primer set including SEQ ID NO: 44 to SEQ ID NO: 59 for detecting FluB. FIG. 5 shows partial cDNA sequence of FluB (SEQ ID NO: 70, which is from position 288 to position 847 of GenBank: MK715610.1) and annealing sites of the primers targeting the M gene of FluB. In some embodiments, a few nucleotides of the primers in Table 3 are adjusted (e.g. point mutations) to be applied for different FluB variants and thus are not completely complementary to the MK715610.1 sequence.

TABLE 3

| | | | SEQ |
|---|---|---|---|
| | Primer | | ID |
| Target | Name | Primer Sequence (5′ to 3′) | NO. |
| FluB | B-FIP | TCTGCCAGCTTTTGGACGCAGATGGTCTCAGCTATGAACAC | 44 |
| | B-BIP | AGAGCTGCAAAGCAACATTGGCTTCCCCATTCTTTTGACTTG | 45 |
| | B-F3 | TTCAGTGCCTGGAGTGAG | 46 |
| | B-B3 | CCATTACATCCTTTGCAATCC | 47 |
| | B-LF | TTTCCCATTCCATTCATTGTT | 48 |
| | B-LB | AGTGCTGAGATCTCTTGG | 49 |
| | B-F4 | AGCAGAGCAGCGAGA | 50 |
| | B-F5 | GCATCACATTCACACAGG | 51 |
| | B-F6 | TCTGTGCTTTATGCGAGAAACA | 52 |
| | B-F7 | ATTCAATGCAAGTAAAACTAGGA | 53 |
| | B-F8 | TGGTCATGTACCTGAATCC | 54 |
| | B-B4 | CATGGAGCTCTGCTTTAG | 55 |
| | B-B5 | TCACAAGAGCTGAATTTCC | 56 |
| | B-B6 | GGTTCGAGCATTATAGATATTTC | 57 |
| | B-B7 | AAAGAACAAATTGAAAGAATATGAAAT | 58 |
| | B-B8 | ATGAAATGGAGAGCTGATAAG | 59 |

The third primer set for detecting FluB basically includes B-FIP (SEQ ID NO: 44), B-BIP (SEQ ID NO: 45), B-F3 (SEQ ID NO: 46), B-B3 (SEQ ID NO: 47), B-LF (SEQ ID NO: 48) and B-LB (SEQ ID NO: 49) targeting the M gene of FluB. In an embodiment, the third primer set further includes additional outer primers B-F4 (SEQ ID NO: 50), B-F5 (SEQ ID NO: 51), B-F6 (SEQ ID NO: 52), B-F7 (SEQ ID NO: 53), B-F8 (SEQ ID NO: 54), B-B4 (SEQ ID NO: 55), B-B5 (SEQ ID NO: 56), B-B6 (SEQ ID NO: 57), B-B7 (SEQ ID NO: 58) and B-B8 (SEQ ID NO: 59) targeting the M gene of FluB.

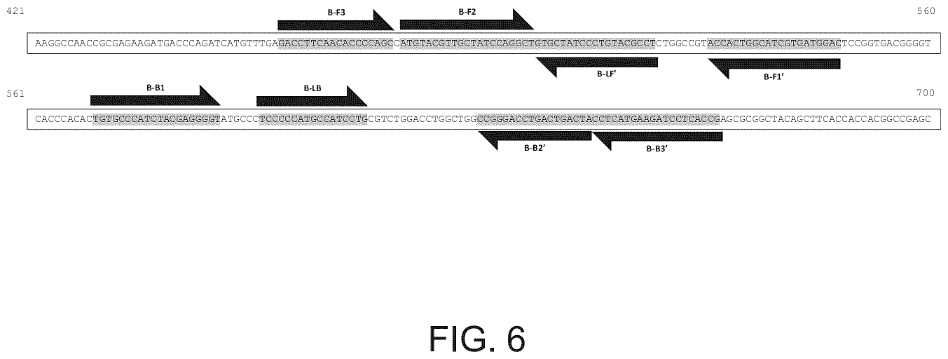

In an embodiment, the primer kit of the LAMP reaction mixture further includes an internal control primer set which includes multiple LAMP primers targeting a housekeeping gene named β-actin in human nasal sample. The method for detecting β-actin is based on the RT-LAMP technology, including the reverse transcription of converting the β-actin RNA from nasal sample to a complementary DNA (cDNA) and then the isothermal amplification whereby the cDNA are amplified with the internal control primer set. Table 4 lists the internal control primer set including SEQ ID NO: 60 to SEQ ID NO: 65 for detecting β-actin. FIG. 6 shows partial cDNA sequence of β-actin (SEQ ID NO: 71, which is from position 421 to position 700 of NCBI Reference Sequence: NM_001101.5) and annealing sites of the primers targeting the β-actin gene.

TABLE 4

| | | | SEQ |
|---|---|---|---|
| | Primer | | ID |
| Target | Name | Primer Sequence (5′ to 3′) | NO. |
| β-actin | IC-FIP | GTCCATCACGATGCCAGTGGTATGTACGTTGCTATCCAGGCT | 60 |
| | IC-BIP | TGTGCCCATCTACGAGGGGTTAGTCAGTCAGGTCCCGG | 61 |
| | IC-F3 | GACCTTCAACACCCCAGC | 62 |
| | IC-B3 | CGGTGAGGATCTTCATGAGG | 63 |
| | IC-LF | AGGCGTACAGGGATAGCAC | 64 |
| | IC-LB | TCCCCCATGCCATCCTG | 65 |

The internal control primer set for detecting β-actin basically includes IC-FIP (SEQ ID NO: 60), IC-BIP (SEQ ID NO: 61), IC-F3 (SEQ ID NO: 62), IC-B3 (SEQ ID NO: 63), IC-LF (SEQ ID NO: 64) and IC-LB (SEQ ID NO: 65) targeting the β-actin gene.

In an embodiment, the LAMP reaction mixture including the primer kit, the enzyme and dNTPs is lyophilized ready-to-use recipe which allows the LAMP reaction mixture to be freeze-dried and stable in room temperature. The recipe is able to be lyophilized inside a strip tube to form a lyo-cake or using a bead dispensing machine to form a lyo-bead without changing the composition.

In an embodiment, the pH indicating dye in the colorimetric LAMP reaction mixture is in a concentration ranged 0.08 to 0.3 mM, and the lyoprotectant sugar in a concentration ranged 1 to 10% (w/v). The lyoprotectant sugar is selected from the group consisting of trehalose, raffinose, dextran, mannitol and mixtures thereof. For example, the pH indicating dye is phenol red or neutral red, but not limited thereto.

In an embodiment, the colorimetric LAMP reaction mixture includes 2 to 7.5% (w/v) of trehalose and 2 to 7.5% (w/v) of raffinose as the lyoprotectant.

In an embodiment, the colorimetric LAMP reaction mixture includes 2 to 7.5% (w/v) of trehalose, 2 to 7.5% (w/v) of raffinose, 1 to 2.5% (w/v) of dextran, and 1 to 5% (w/v) of mannitol as the lyoprotectant.

In an embodiment, a magnesium salt, such as magnesium sulfate, in a concentration ranged 4 to 12 mM is further included in the colorimetric LAMP reaction mixture. The magnesium salt facilitates the primer to target and bind tightly to the target.

In an embodiment, an intercalating fluorescence dye, such as EvaGreen® dye, is further added into the colorimetric LAMP reaction mixture for quality control or detection for fluorescence LAMP.

The present disclosure also provides the colorimetric LAMP-friendly direct lysis buffer. The direct lysis buffer allows direct amplification without further purification after the viruses are lysed and the genomic materials are released into the buffer, so that the total assay time is reduced. The present disclosure provides a recipe of the direct lysis buffer to release the genomic materials of the viruses, and the lysis buffer harboring the genomic materials can be directly added to the above-mentioned colorimetric LAMP reaction mixture without further purification. The direct lysis buffer of the present disclosure utilizes an optimized buffer composition including detergents and LAMP tolerable chemicals to achieve virus lysis and amplification.

In an embodiment, the direct lysis buffer of the present disclosure includes potassium chloride (KCl) in a concentration ranged 10 to 50 mM, ammonium sulfate [(NH₄)₂SO₄] in a concentration ranged 10 to 50 mM, and a detergent in a concentration ranged 0.5 to 6% (w/v). The detergent is selected from the group consisting of 2-ethylhexan-1-ol;2-methyloxirane;oxirane (Ecosurf™ EH-9, CAS number: 64366-70-7), secondary alcohol ethoxylate (Tergitol™ Type 15 S-7 or Type 15 S-9, CAS number: 84133-50-6), 2-[4-(2,4,4-trimethylpentan-2-yl)phenoxy]ethanol (Triton® X-100, CAS number: 9002-93-1), 2-[4-(2,4,4-trimethylpentan-2-yl)phenoxy]ethan-1-ol (IGEPAL®, CAS number: 9002-93-1), and {2-[3,4-bis(2-hydroxyethoxy)oxolan-2-yl]-2-(2-hydroxyethoxy)ethoxy}ethyl dodecanoate (Tween® 20, CAS number: 9005-64-5) and mixtures thereof. This composition makes up the direct lysis buffer friendly to the colorimetric LAMP reaction with capability to release the genomic materials from SARS-CoV-2, FluA and FluB.

In an embodiment, the direct lysis buffer includes 10 to 50 mM of potassium chloride, 10 to 50 mM of ammonium sulfate, and 0.5 to 6% (w/v) of Ecosurf™ EH-9.

In an embodiment, the direct lysis buffer includes 10 to 50 mM of potassium chloride, 10 to 50 mM of ammonium sulfate, and 0.5 to 6% (w/v) of Tergitol™ Type 15 S-7.

In an embodiment, the direct lysis buffer includes 10 to 50 mM of potassium chloride, 10 to 50 mM of ammonium sulfate, and 0.5 to 6% (w/v) of Tergitol™ Type 15 S-9.

In an embodiment, the direct lysis buffer includes 10 to 50 mM of potassium chloride, 10 to 50 mM of ammonium sulfate, and 0.5 to 6% (w/v) of Triton® X-100.

In an embodiment, the direct lysis buffer includes 10 to 50 mM of potassium chloride, 10 to 50 mM of ammonium sulfate, and 0.5 to 6% (w/v) of IGEPAL®.

In an embodiment, the direct lysis buffer includes 10 to 50 mM of potassium chloride, 10 to 50 mM of ammonium sulfate, and 0.5 to 6% (w/v) of Tween® 20.

An illustrative recipe of the LAMP assay system of the present disclosure is shown in Table 5. The LAMP reaction is conducted in 65° C. for 20 minutes (30 seconds per cycle, repeat for 40 cycles).

TABLE 5

| Reagent | Concentration | |
|---|---|---|
| dH₂O | Top Up to 25 μl | |
| dNTPs | 1.12 | mM |
| Primer | 1x | |
| Fluorescence dye | 0.6x | |
| DNA Enzyme | 9.6 | units |
| RT Enzyme | 3 | units |
| Mg²⁺ | 8 | mM |
| Lysis buffer 1 | 1x | |
| Lysis buffer 2 | 2% | |
| pH Indicating dye | 0.08 | mM |
| Lyoprotectant 1 | 1.9% | |
| Lyoprotectant 2 | 1.9% | |
| Template | 10.00 | μl |

In Table 5, the lysis buffer 1 may include potassium chloride and ammonium sulfate in 10× concentration, and the lysis buffer 2 may include Ecosurf™ EH-9, Tergitol™ Type 15 S-7, Tergitol™ Type 15 S-9, Triton® X-100, IGEPAL®, or Tween® 20. For example, the lysis buffer 2 includes Tergitol™ Type 15 S-9. The lyoprotectants 1 and 2 may include trehalose, raffinose, dextran or mannitol. For example, the lyoprotectant 1 includes dextran and the lyoprotectant 2 includes mannitol.

The present disclosure utilizes the modified LAMP with intercalating fluorescence dye and/or pH Indicating dye for rapid, sensitive and specific diagnosis of SARS-CoV-2, FluA and FluB infections in humans via fluorescence or naked eye. The following are examples illustrating the applications of the LAMP assay system in the present disclosure for detection of SARS-CoV-2, FluA and FluB. The above-mentioned primer sets are optimized for the rapid detection of SARS-CoV-2, FluA and FluB through the modified LAMP conducted in 65° C. for 20 minutes.

Example 1 illustrates the comparison of the fluorescence LAMP with the colorimetric LAMP using SARS-CoV-2, FluA H1N1 and FluB reference RNA. The colorimetric LAMP reaction mixture mentioned above and the pH indicating dye of phenol red were used for the colorimetric LAMP assay. For the phenol red, the color of the reaction mixture turns orange/yellow for the positive sample or remains pink for the negative sample after amplification. An intercalating fluorescence dye was added into the recipe for the fluorescence LAMP assay. The first primer set in Table 1, the second primer set in Table 2, and the third primer set in Table 3 were used for detection of SARS-CoV-2, FluA and FluB.

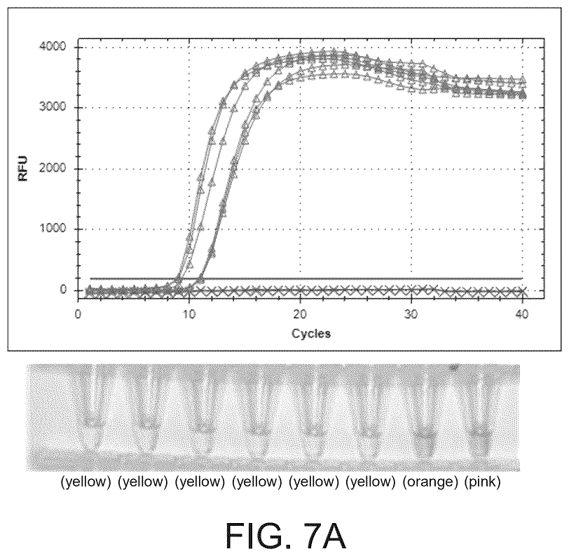

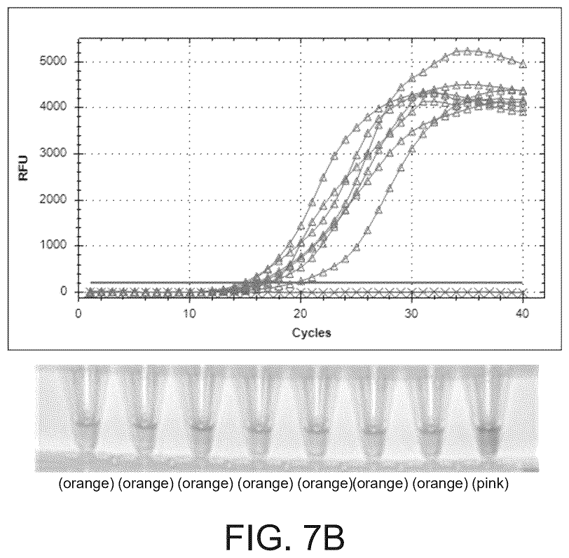

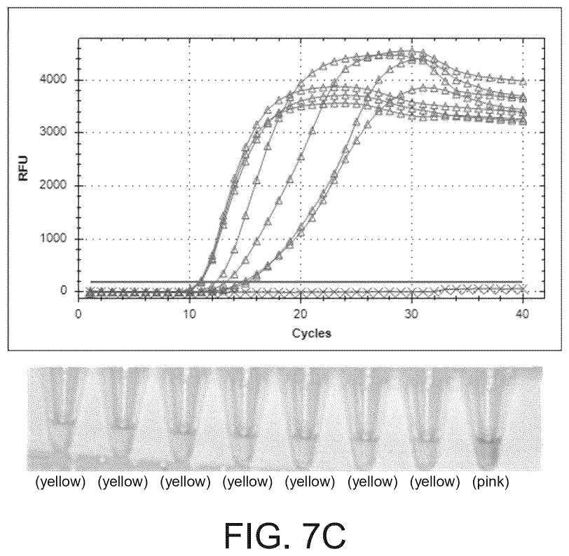

FIG. 7A shows the comparison of the fluorescence LAMP with the colorimetric LAMP using SARS-CoV-2 reference RNA, FIG. 7B shows the comparison of the fluorescence LAMP with the colorimetric LAMP using FluA H1N1 reference RNA, and FIG. 7C shows the comparison of the fluorescence LAMP with the colorimetric LAMP using FluB reference RNA. The upper parts of FIGS. 7A to 7C show the amplification curves of the fluorescence LAMP assays, while the lower parts of FIGS. 7A to 7C show the visual results of the colorimetric LAMP assay. As shown in FIGS. 7A to 7C, the color changes of the colorimetric LAMP were in concordance to the amplification results of the fluorescence LAMP. The duration of the colorimetric LAMP reaction was 20 minutes which was equivalent to 40 Cq in the amplification cycle.

Example 2 illustrates the performance of the additional outer primers on SARS-CoV-2, FluA and FluB assay with the lyophilized colorimetric LAMP reaction mixture of the present disclosure. The assay was conducted using SARS-CoV-2, FluA H1N1 and H3N2, and FluB/Brisbane reference materials. As mentioned above, the first primer set for SARS-CoV-2 detection may include the additional outer primers of S-OF4 to S-OF6, S-OB4 to S-OB6, S-HF4 to S-HF6, and S-HB4 to S-HB6, the second primer set for FluA detection may include the additional outer primers of A-6B4 to A-6B10, and the third primer set for FluB detection may include the additional outer primers of B-F4 to B-F8 and B-B4 to B-B8.

Tables 6A to 6C show the SARS-CoV-2 assay with the additional outer primers, using three different commercial SARS-CoV-2 materials, respectively. The results show that the performance of SARS-CoV-2 assay with the additional outer primers added with overall 2-6 Cq improved and higher detection rate and consistency in lower input (50 cp/rxn and 25 cp/rxn). Therefore, with the additional outer primers for SARS-CoV-2 assay, the amplification speed and the detection limit were improved.

TABLE 6A

| | | | Commercial SARS-CoV-2 material 1 | | | | | |
|---|---|---|---|---|---|---|---|---|
| | | | | | | With additional | | |
| | | | Original 6 primers | | | outer primers | | |
| LoD | cp/rxn | cp/ml | Cq | Std Dev | Rate | Cq | Std Dev | Rate |
| 4x LoD | 100 | 4000 | 14.51 | 1.246 | 4/4 | 12.57 | 0.406 | 4/4 |
| 2x LoD | 50 | 2000 | 14.87 | 2.162 | 4/4 | 13.21 | 0.344 | 4/4 |
| 1x LoD | 25 | 1000 | 14.97 | 1.77 | 4/4 | 14.2 | 2.427 | 4/4 |
| 0x LoD | 0 | 0 | 0 | 0 | 0/4 | 0 | 0 | 0/4 |

TABLE 6B

| | | | Commercial SARS-CoV-2 material 2 | | | | | |
|---|---|---|---|---|---|---|---|---|
| | | | | | | With additional | | |
| | | | Original 6 primers | | | outer primers | | |
| LoD | cp/rxn | cp/ml | Cq | Std Dev | Rate | Cq | Std Dev | Rate |
| 4x LoD | 100 | 4000 | 17.21 | 1.287 | 4/4 | 13.94 | 1.32 | 4/4 |
| 2x LoD | 50 | 2000 | 20.18 | 11.125 | 3/4 | 15.96 | 3.859 | 4/4 |
| 1x LoD | 25 | 1000 | 16.36 | 3.191 | 2/4 | 13.83 | 2.537 | 4/4 |
| 0x LoD | 0 | 0 | 0 | 0 | 0/4 | 0 | 0 | 0/4 |

TABLE 6C

| | | | Commercial SARS-CoV-2 material 3 | | | | | |
|---|---|---|---|---|---|---|---|---|
| | | | | | | With additional | | |
| | | | Original 6 primers | | | outer primers | | |
| LoD | cp/rxn | cp/ml | Cq | Std Dev | Rate | Cq | Std Dev | Rate |
| 4x LoD | 100 | 4000 | 15.68 | 2.114 | 4/4 | 13.46 | 0.768 | 4/4 |
| 2x LoD | 50 | 2000 | 16.79 | 1.246 | 3/4 | 13.88 | 0.903 | 4/4 |
| 1x LoD | 25 | 1000 | 22.06 | 9.811 | 2/4 | 16.16 | 3.07 | 4/4 |
| 0x LoD | 0 | 0 | 0 | 0 | 0/4 | 0 | 0 | 0/4 |

Tables 7A and 7B show the FluA assay with the additional outer primers, using FluA H1N1 and FluA H3N2 materials, respectively. The results show that the performance of FluA assay with the additional outer primers added with overall 1-2 Cq improved. Therefore, with the additional outer primers for FluA assay, the amplification speed was improved.

TABLE 7A

| | | FluA H1N1 | | | | | |
|---|---|---|---|---|---|---|---|
| | | Original 6 primers | | | With additional outer primers | | |
| cp/rxn | cp/ml | Cq | Std Dev | Rate | Cq | Std Dev | Rate |
| 192 | 7680 | 19.69 | 4.496 | 3/3 | 17.39 | 1.915 | 3/3 |
| 96 | 3840 | 23.14 | 4.628 | 3/3 | 17.57 | 1.442 | 3/3 |
| 48 | 1920 | 25.52 | 10.102 | 3/3 | 21.29 | 3.990 | 3/3 |
| 0 | 0 | 0.00 | 0.00 | 0/3 | 0.00 | 0.00 | 0/3 |

TABLE 7B

| | | FluA H3N2 | | | | | |
|---|---|---|---|---|---|---|---|
| | | Original 6 primers | | | With additional outer primers | | |
| cp/rxn | cp/ml | Cq | Std Dev | Rate | Cq | Std Dev | Rate |
| 192 | 7680 | 24.68 | 11.236 | 3/3 | 16.97 | 0.904 | 3/3 |
| 96 | 3840 | 19.20 | 3.271 | 3/3 | 17.16 | 1.209 | 3/3 |
| 48 | 1920 | 26.51 | 10.367 | 2/3 | 18.21 | 1.719 | 2/3 |
| 0 | 0 | 0.00 | 0.00 | 0/3 | 0.00 | 0.00 | 0/3 |

Table 8 shows the FluB assay with the additional outer primers, using FluB/Brisbane material. The results show that the performance of FluB assay with the additional outer primers added with overall 0.5-2 Cq improved and higher detection rate and consistency in lower input (3.75 cp/rxn). Therefore, with the additional outer primers for FluB assay, the amplification speed and the detection limit were improved.

TABLE 8

| | | | FluB | | | | | |
|---|---|---|---|---|---|---|---|---|
| | | | | | | With additional | | |
| | | | Original 6 primers | | | outer primers | | |
| | | | | Std | | | Std | |
| LoD | cp/rxn | cp/ml | Cq | Dev | Rate | Cq | Dev | Rate |
| 256 | 3840 | 153600 | 10.1 | 0.011 | 3/3 | 9.65 | 0.114 | 3/3 |
| 64 | 960 | 38400 | 10.38 | 0.204 | 3/3 | 10.25 | 0.026 | 3/3 |
| 16 | 240 | 9600 | 11.11 | 0.22 | 3/3 | 10.88 | 0.169 | 3/3 |
| 4 | 60 | 2400 | 12.42 | 0.437 | 3/3 | 11.79 | 0.228 | 3/3 |
| 1 | 15 | 600 | 14.85 | 2.212 | 3/3 | 12.86 | 0.114 | 3/3 |
| 0.5 | 7.5 | 300 | 14.14 | 0.321 | 3/3 | 14.81 | 2.044 | 3/3 |
| 0.25 | 3.75 | 150 | 15.9 | 0.867 | 2/3 | 15.3 | 1.753 | 3/3 |
| 0 | 0 | 0 | 0 | 0.00 | 0/3 | 0 | 0.00 | 0/3 |

From Table 6A to Table 8, the assay with the additional outer primers improves the speed of the assay for 1-6 Cq (3 minutes) and the detection limit of the assay. Therefore, the designed primer sets with the additional outer primers of the present disclosure for SARS-CoV-2, FluA and FluB detection improve the overall amplification speed and the sensitivity of the assay.

The colorimetric LAMP reaction mixture of the present disclosure utilizes an optimized buffer composition, resulting in a rapid diagnosis turnaround time of 20 minutes. All these are achieved without compromising on the sensitivity and specificity of SARS-CoV-2, FluA and FluB detection.

In addition, for the primer buffer, the present disclosure achieves rapid fluorescence signal generation and color change with reduction of Tris concentration to 0.5 mM to 5 mM to allow color change from pinkish to yellowish or yellowish to pinkish.

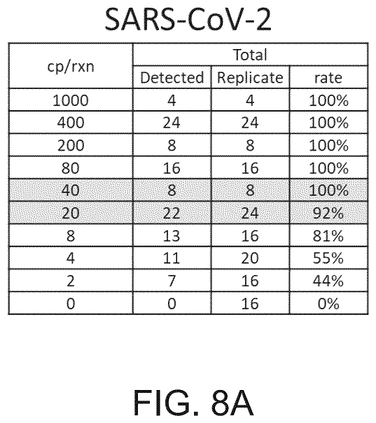

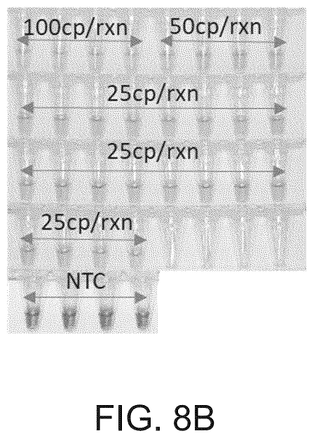

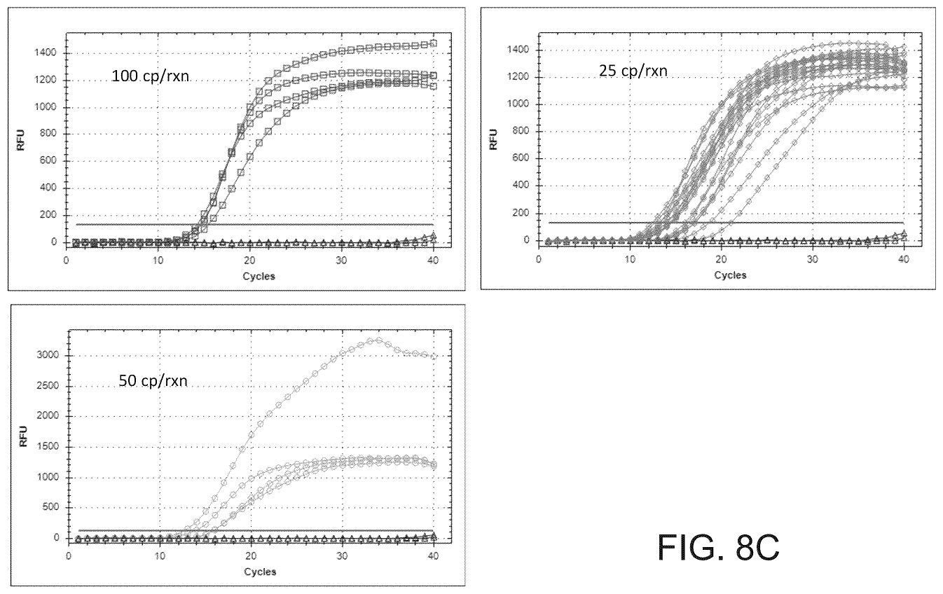

Example 3 illustrates the limit of detection (LoD) of SARS-CoV-2 assay using SARS-CoV-2 reference materials on the lyophilized colorimetric LAMP reaction mixture of the present disclosure. FIG. 8A shows the summary detection rate of different sample inputs for SARS-CoV-2 assay, while FIG. 8B and FIG. 8C show the amplification results in color change and fluorescence signal, respectively, of different sample inputs for SARS-CoV-2 assay. As a result, the assay is capable of detecting down to just 20 to 40 copies of SARS-CoV-2 within 20 minutes. The limit of detection (LoD) of SARS-CoV-2 is thus predicted to be around 25 copies per reaction which equivalent to 1000 copies per ml of initial input material. FIG. 8B and FIG. 8C also revealed that 20 out of 20 replicates were detected with 25 copies/rxn.

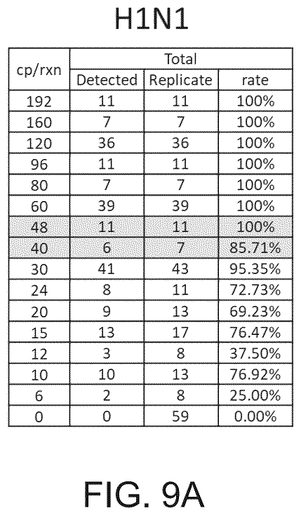

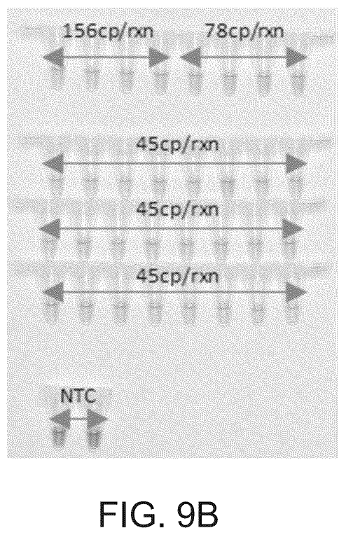

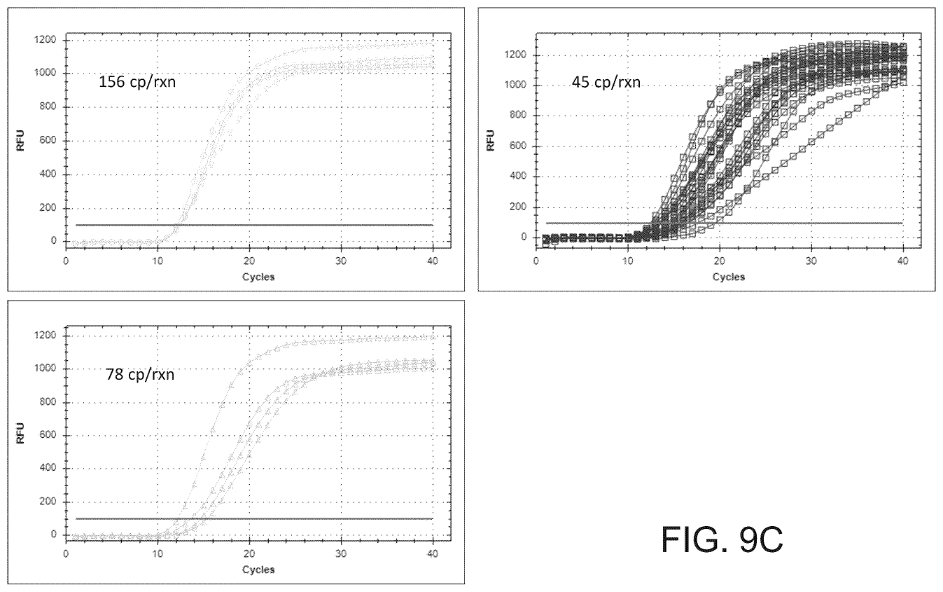

Example 4 illustrates the limit of detection (LoD) of FluA assay using FluA H1N1 full length reference RNA on the lyophilized colorimetric LAMP reaction mixture of the present disclosure. FIG. 9A shows the summary detection rate of different sample inputs for FluA assay, while FIG. 9B and FIG. 9C show the amplification results in color change and fluorescence signal, respectively, of different sample inputs for FluA assay. As a result, the assay is capable of detecting down to just 40 to 48 copies of FluA within 20 minutes. The limit of detection (LoD) of FluA is thus predicted to be around 45 copies per reaction which equivalent to 1800 copies per ml of initial input material. FIG. 9B and FIG. 9C also revealed that 24 out of 24 replicates were detected with 45 copies/rxn.

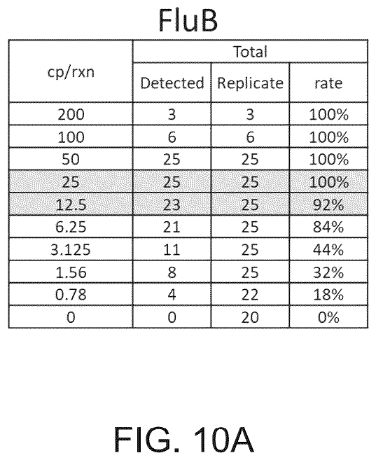

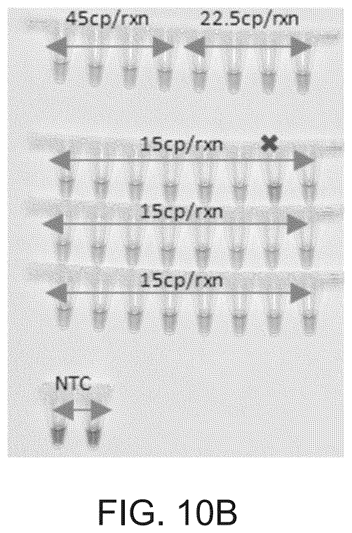

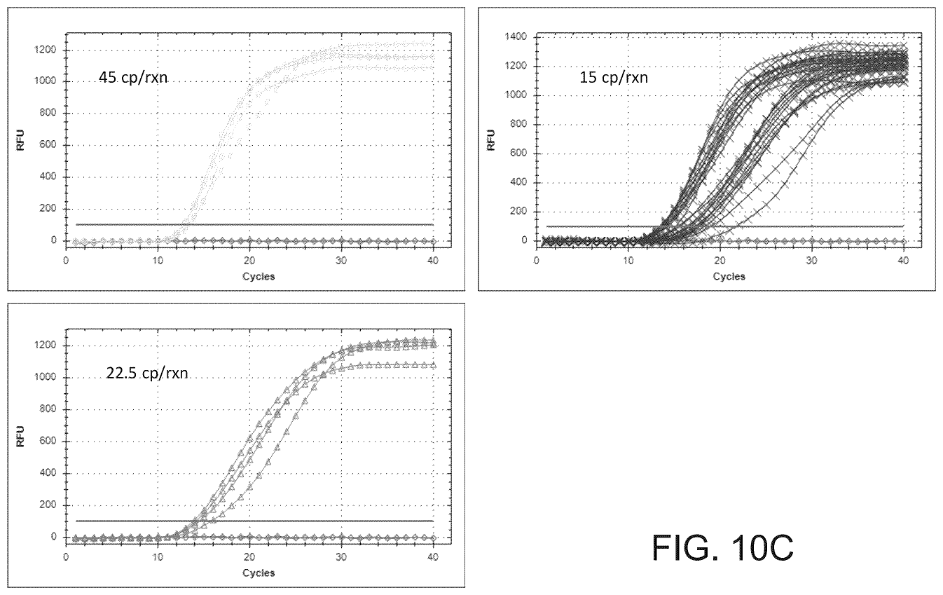

Example 5 illustrates the limit of detection (LoD) of FluB assay using FluB reference RNA on the lyophilized colorimetric LAMP reaction mixture of the present disclosure. FIG. 10A shows the summary detection rate of different sample inputs for FluB assay, while FIG. 10B and FIG. 10C show the amplification results in color change and fluorescence signal, respectively, of different sample inputs for FluB assay. As a result, the assay is capable of detecting down to just 12.5 to 25 copies of FluB within 20 minutes. The limit of detection (LoD) of FluB is thus predicted to be around 15 copies per reaction which equivalent to 600 copies per ml of initial input material. FIG. 10B and FIG. 10C also revealed that 23 out of 24 replicates were detected with 15 copies/rxn.

Example 6 illustrates the in-silico analysis of SARS-CoV-2 assay with GISAID database, and Table 9 shows the capturing rate of SARS-CoV-2 primers via in-silico analysis. The SARS-CoV-2 assay with the 24 primers in Table 1 were checked with GISAID database. The analysis result showed that the designed primers are matched with the SARS-CoV-2 variant of concern (VOC) in-silico, and are able to capture 99.8% of SARS-CoV-2 (n=366707) and 98.2% on Omicron variant.

TABLE 9

| | GISAID | | | | | |
|---|---|---|---|---|---|---|
| | SARS-CoV-2 (from 2023 Jan. 1) | | | | | |
| | Total Sequences | | | | | |
| | 366707 (total) | | | 360162 (Omicron) (98.2%) | | |
| | | Total | | | Total | |
| LAMP | Perfect | align-able | | Perfect | align-able | |
| primers | match | sequence | Ratio*¹ | match | sequence | Ratio*¹ |
| All*² | 344516 | 345030 | 0.9985 | 339101 | 339588 | 0.9986 |
| All*³ | 344391 | 345030 | 0.9981 | 338985 | 339588 | 0.9982 |

*¹Ratio = No. of perfect match/No. of total align-able match
*²Perfect match and single point mutation of BIP, FIP, B3, F3
*³All single point mutation

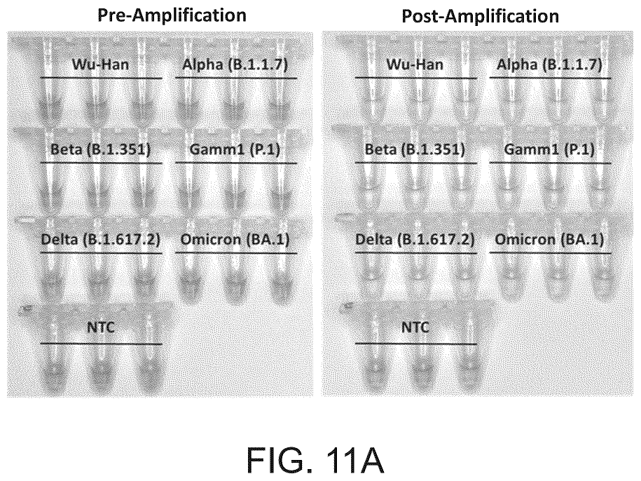

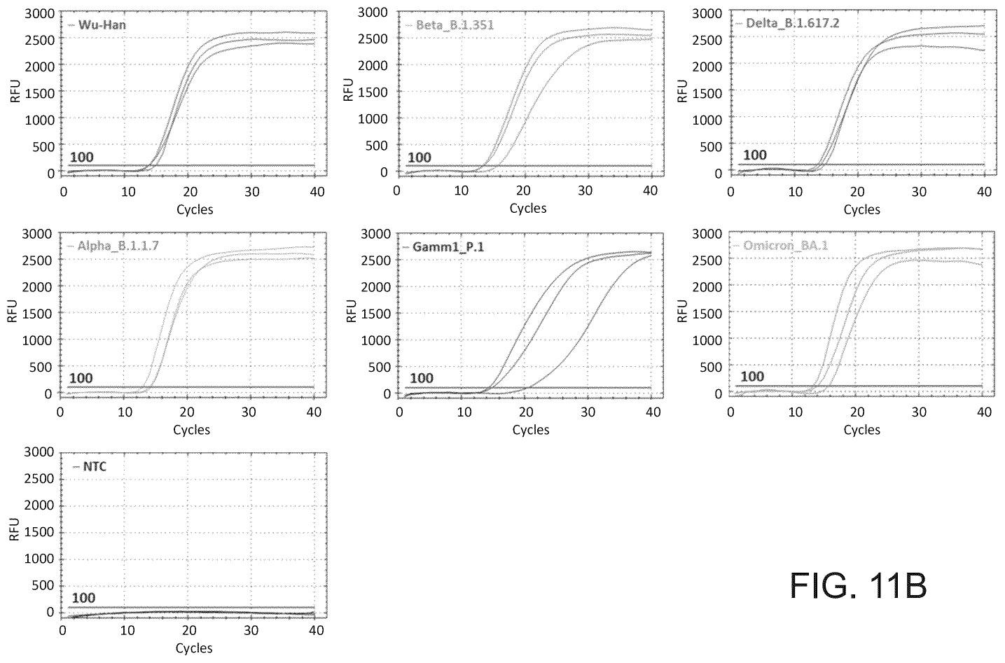

Example 7 illustrates the sensitivity analysis for the SARS-CoV-2 primers of the present disclosure. To ascertain the sensitivity of the primers for detecting SARS-CoV-2, the primers S-OFIP, S-OBIP, S-OLF, S-OLB, S-OF3 to S-OF6, S-OB3 to S-OB6, S-HFIP, S-HBIP, S-HLF, S-HLB, S-HF3 to S-HF6, and S-HB3 to S-HB6 were tested with reference materials of SARS-CoV-2 variant of concern (VOC). The assay was conducted using 100 cp/rxn input which is equivalent to 4× LoD. FIG. 11A and FIG. 11B show the color change and the fluorescence signal detected by SARS-CoV-2 assay while using various SARS-CoV-2 strains. The result shows that the designed primers are able to detect all the SARS-CoV-2 VOC in low input concentration, suggesting high sensitivity for the SARS-CoV-2 primers of the present disclosure.

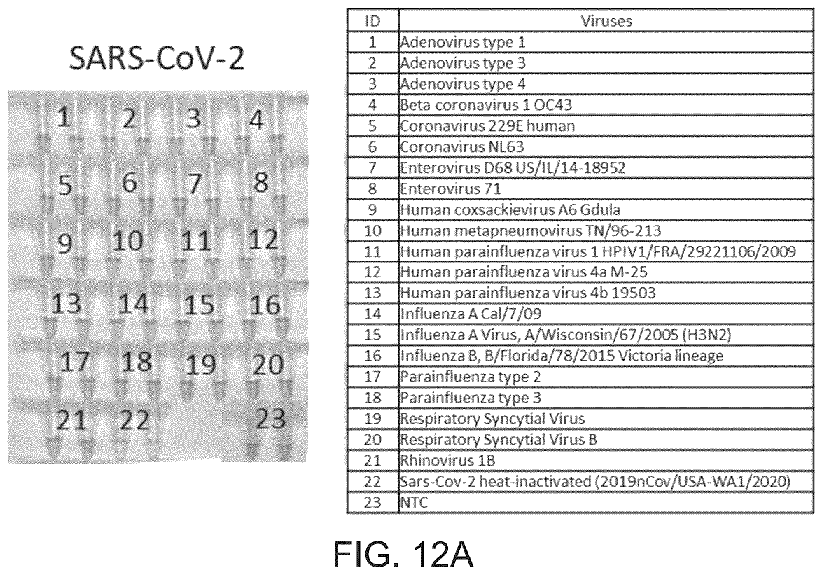

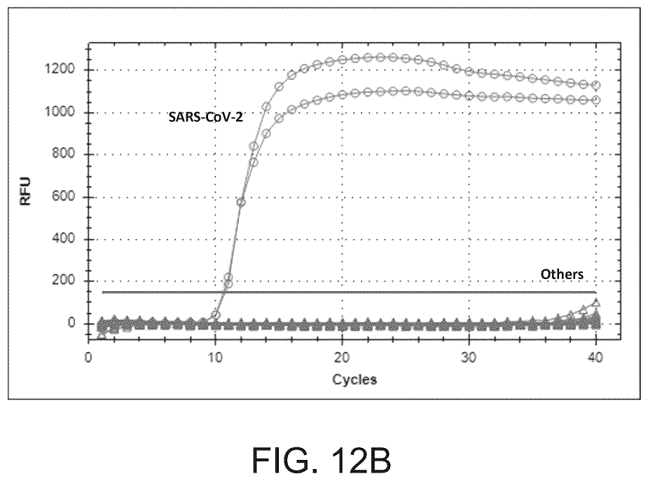

Example 8 illustrates the specificity analysis for the SARS-CoV-2 primers of the present disclosure. To ascertain the specificity of the primers for detecting SARS-CoV-2, the primers S-OFIP, S-OBIP, S-OLF, S-OLB, S-OF3 to S-OF6, S-OB3 to S-OB6, S-HFIP, S-HBIP, S-HLF, S-HLB, S-HF3 to S-HF6, and S-HB3 to S-HB6 were checked with NCBI BLAST, and the blast result shows that no other similar species have 100% same fragment compared to each of the primers of the present disclosure. The specificity analysis was conducted using the designed primers with different respiratory pathogens. The assay was conducted using high copy of challenging pathogens with 100 cp/rxn input which is equivalent to 4× LoD of reference SARS-CoV-2 materials. FIG. 12A and FIG. 12B show the specificity analysis of SARS-CoV-2 assay with different respiratory pathogens. The results show that only the virus of No. 22 was amplified. In other words, the designed SARS-CoV-2 primers of the present disclosure can be only used to amplify and detect SARS-CoV-2 2-ORMT and Helicase genes, suggesting high specificity for the SARS-CoV-2 primers of the present disclosure.

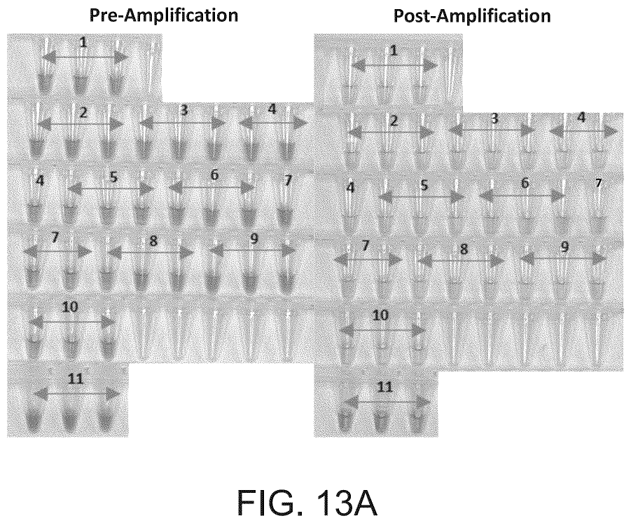

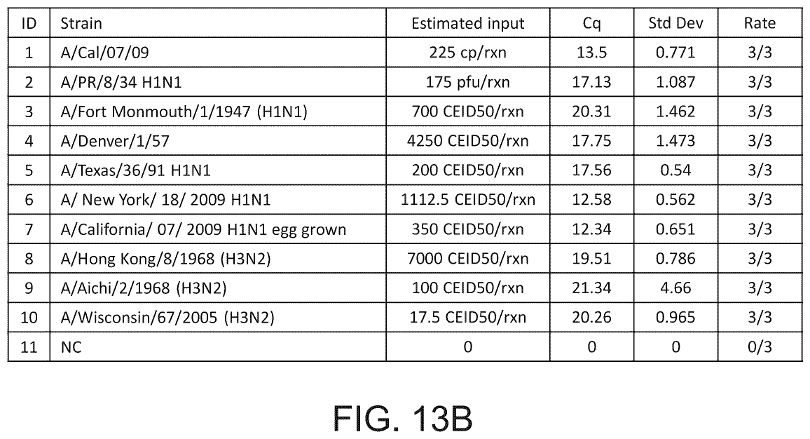

Example 9 illustrates the sensitivity analysis for the FluA primers of the present disclosure. To ascertain the sensitivity of the primers for detecting FluA, the primers A-6FIP, A-6BIP, A-6LF, A-6LB, A-6F3, A-6B3 to A-6B10, A-5FIP, A-5BIP, A-5LF, A-5LB, A-5F3 and A-5B3 were tested with reference materials of FluA H1N1, H3N2, H5N7 and H7N9. The assay was conducted using various inputs of FluA virus strains. Table 10 shows the sensitivity analysis of the FluA assay against different FluA subtypes, revealing that the assay is able to detect H1N1, H3N2, H5N1 and H7N9 with different sensitivity. FIG. 13A and FIG. 13B show the color change and the fluorescence signal detected by FluA assay while using various FluA H1 and H3 strains. The result shows that the designed primers are able to detect all FluA strains with different sensitivity, suggesting high sensitivity for the FluA primers of the present disclosure.

TABLE 10

| Subtype | cp/rxn | cp/ml | Cq | Std Dev | Rate |
|---|---|---|---|---|---|
| H1N1 (Ref) | 225 | 9000 | 13.84 | 1.288 | 4/4 |
| H3N2 | 90 | 3600 | 20.11 | 0.804 | 3/3 |
| H5N1 | 11520 | 460800 | 15.99 | 0.415 | 4/4 |
| | 2880 | 115200 | 16.8 | 0.688 | 4/4 |
| | 720 | 28800 | 19.09 | 1.574 | 4/4 |
| | 360 | 14400 | 25.19 | 4.378 | 2/3 |
| H7N9 | 11520 | 460800 | 15.75 | 0.239 | 4/4 |
| | 2880 | 115200 | 16.86 | 0.323 | 4/4 |
| | 720 | 28800 | 18.41 | 0.343 | 4/4 |
| | 360 | 14400 | 23.4 | 0.953 | 3/3 |
| | 180 | 7200 | 25.59 | 2.933 | 3/3 |
| | 90 | 3600 | 25.44 | 0.739 | 3/3 |
| | 45 | 1800 | 26.92 | 2.641 | 3/3 |
| NTC | 0 | 0 | 0 | 0 | 0/4 |

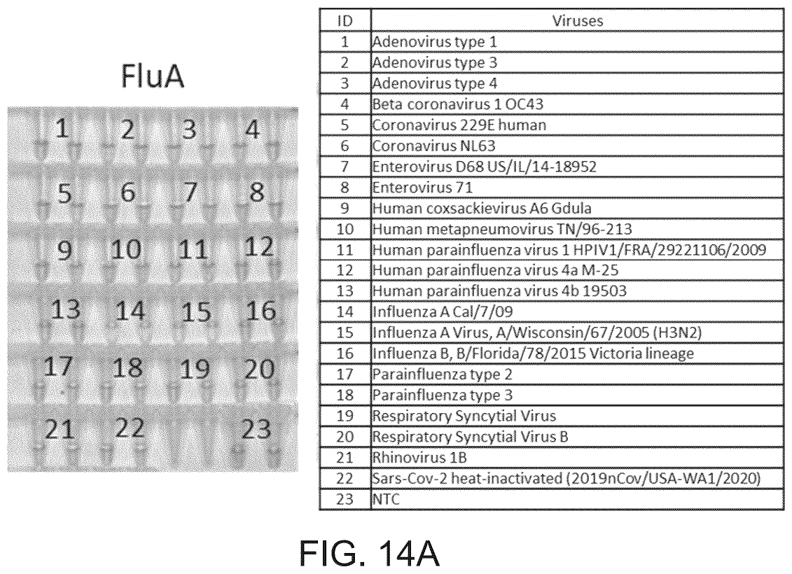

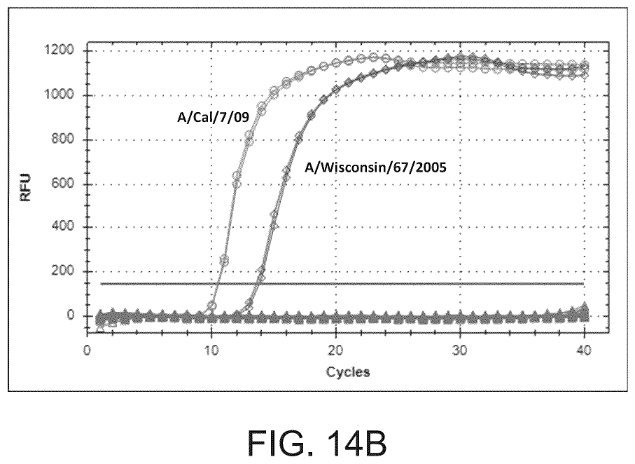

Example 10 illustrates the specificity analysis for the FluA primers of the present disclosure. To ascertain the specificity of the primers for detecting FluA, the primers A-6FIP, A-6BIP, A-6LF, A-6LB, A-6F3, A-6B3 to A-6B10, A-5FIP, A-5BIP, A-5LF, A-5LB, A-5F3 and A-5B3 were checked with NCBI BLAST, and the blast result shows that no other similar species have 100% same fragment compared to each of the primers of the present disclosure. The specificity analysis was conducted using the designed primers with different respiratory pathogens. The assay was conducted using high copy of challenging pathogens with 180 cp/rxn input which is equivalent to 4× LoD of reference FluA H1N1 RNA materials. FIG. 14A and FIG. 14B show the specificity analysis of FluA assay with different respiratory pathogens. The results show that only the viruses of No. 14 (Influenza A Cal/7/09) and No. 15 (Influenza A virus, A/Wisconsin/67/2005) were amplified. In other words, the designed FluA primers of the present disclosure can be only used to amplify and detect FluA Matrix Protein (M) gene, suggesting high specificity for the FluA primers of the present disclosure.

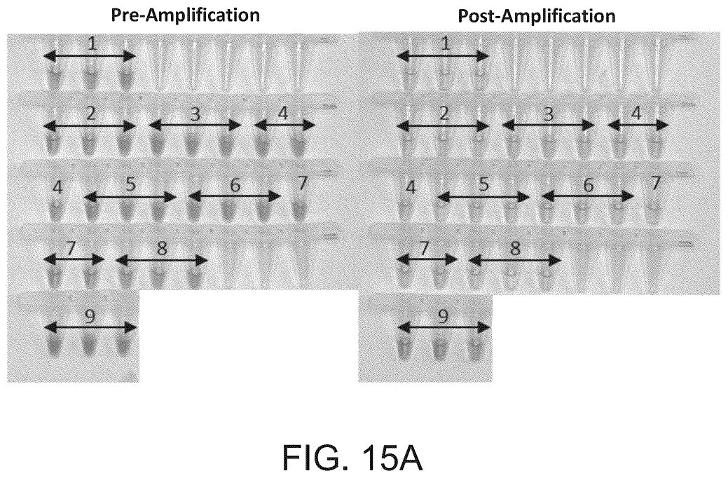

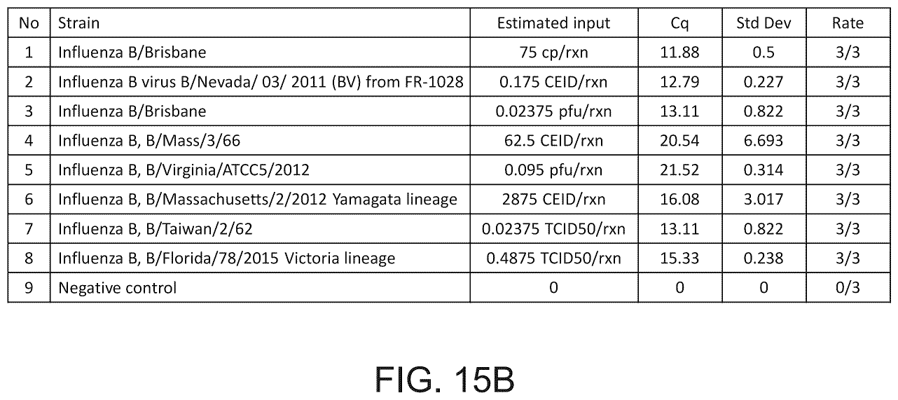

Example 11 illustrates the sensitivity analysis for the FluB primers of the present disclosure. To ascertain the sensitivity of the primers for detecting FluB, the primers B-FIP, B-BIP, B-LF, B-LB, B-F3 to B-F8, B-B3 to B-B8 were tested with reference materials of two FluB lineage namely B/Yamagata and B/Victoria. The assay was conducted using various inputs of FluB virus strains. FIG. 15A and FIG. 15B show the color change and the fluorescence signal detected by FluB assay while using various FluB strains. The result shows that the designed primers are able to detect all FluB strains with different sensitivity, suggesting high sensitivity for the FluB primers of the present disclosure.

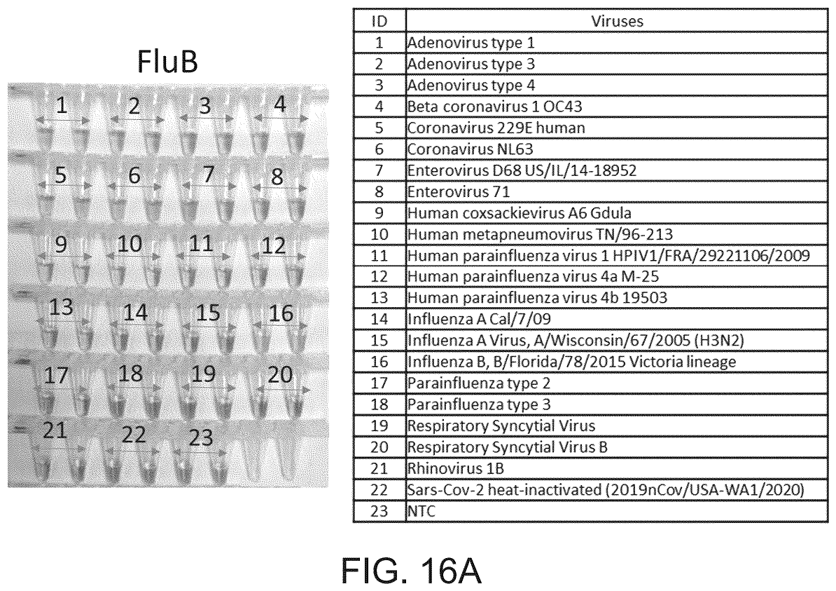

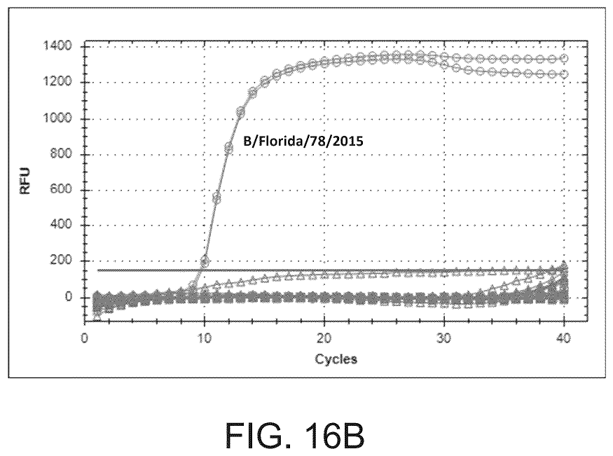

Example 12 illustrates the specificity analysis for the FluB primers of the present disclosure. To ascertain the specificity of the primers for detecting FluB, the primers B-FIP, B-BIP, B-LF, B-LB, B-F3 to B-F8, B-B3 to B-B8 were checked with NCBI BLAST, and the blast result shows that no other similar species have 100% same fragment compared to each of the primers of the present disclosure. The specificity analysis was conducted using the designed primers with different respiratory pathogens. The assay was conducted using high copy of challenging pathogens with 60 cp/rxn input which is equivalent to 4× LoD of reference FluB materials. FIG. 16A and FIG. 16B show the specificity analysis of FluB assay with different respiratory pathogens. The results show that only the virus of No. 16 (Influenza B, B/Florida/78/2015 Victoria lineage) was amplified. In other words, the designed FluB primers of the present disclosure can be only used to amplify and detect FluB Matrix Protein (M) gene, suggesting high specificity for the FluB primers of the present disclosure.

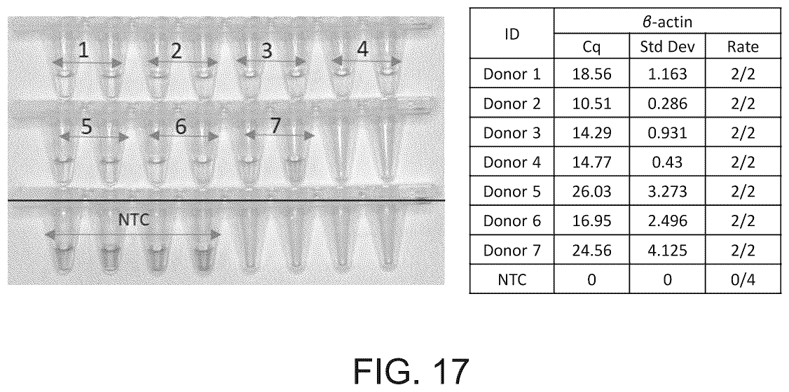

Example 13 illustrates the performance test for the internal control β-actin primers of the present disclosure. To ascertain the performance of the primers for detecting β-actin, the primers IC-FIP, IC-BIP, IC-LF, IC-LB, IC-F3 and IC-B3 were tested with nasal samples collected from multiple human donors. In this assay, the β-actin primers were also incorporated into the lyophilized LAMP reaction mixture of the present disclosure and the nasal samples were collected from various individuals and lysed with the direct lysis buffer of the present disclosure prior the assay. FIG. 17 shows the color change and the fluorescence signal detected by β-actin assay with different donor samples. The result shows that the designed primers are able to detect the β-actin gene across different individuals with slightly different Cq value, suggesting high sensitivity for the β-actin primers of the present disclosure.

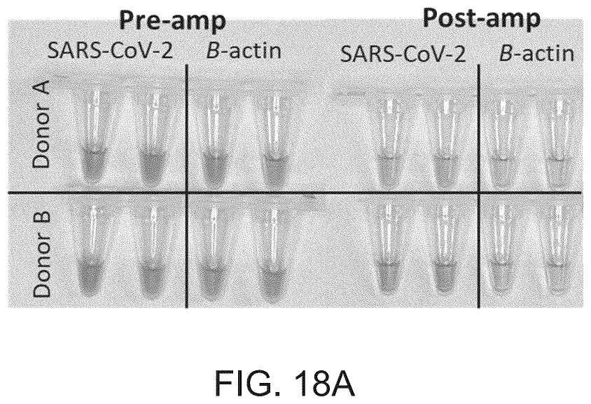

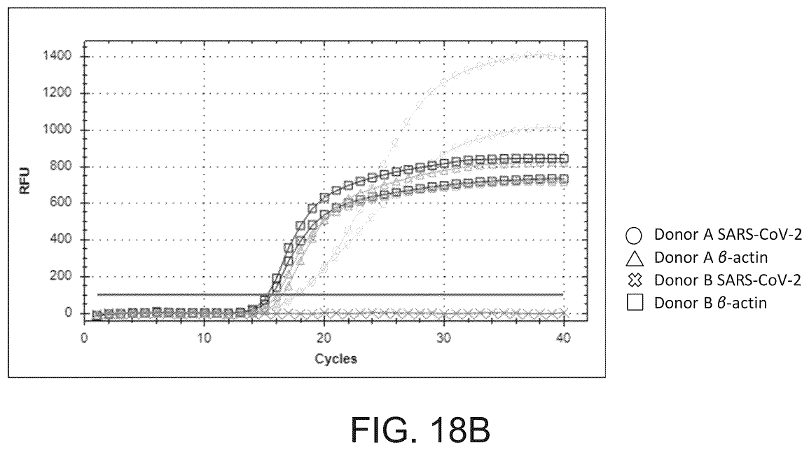

Example 14 illustrates the multiple detection of the respiratory pathogen in the nasal sample with β-actin gene assay served as the internal control without prior purification. The collected nasal sample was directly submerged into the direct lysis buffer of the present disclosure. FIG. 18A and FIG. 18B show the multiple detection of SARS-CoV-2 and β-actin directly from the nasal sample without purification. In this assay, the donor A is SARS-CoV-2 positive sample, and the donor B is SARS-CoV-2 negative sample. The results show that the SARS-CoV-2 target was successfully amplified from the donor A sample, while the internal control β-actin were successfully amplified from both the donor A sample and the donor B sample, suggesting the capability to detect the target RNA directly from the nasal sample with the internal control β-actin served as reference of sample input without prior purification of sample.

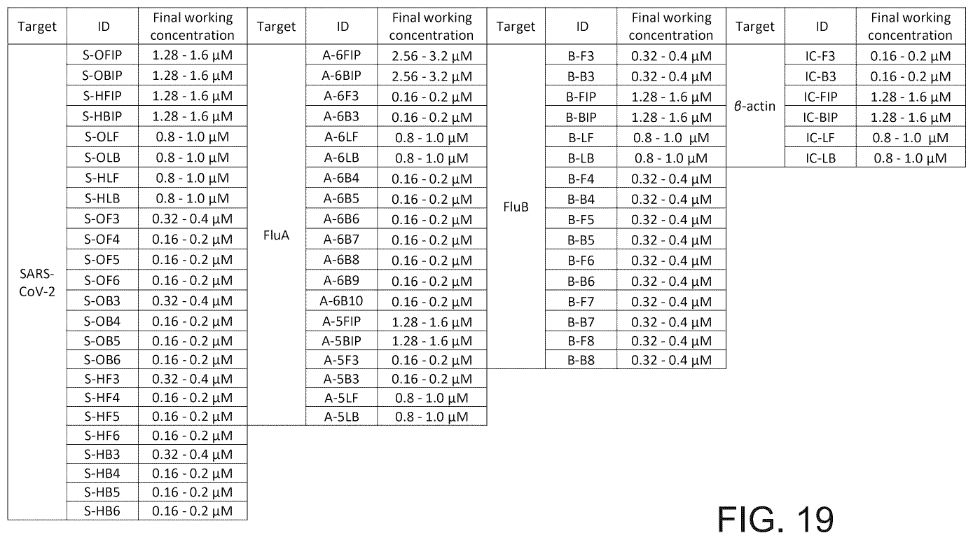

Example 15 illustrates the working concentrations of the LAMP primers for each target used in the fluorescence LAMP and the colorimetric LAMP. The primers were pre-mixed into 10× and 8× primer stocks prior adding into the LAMP reaction mixture for lyophilization. FIG. 19 shows the final working concentrations of the primers for the assays used in the LAMP reaction.

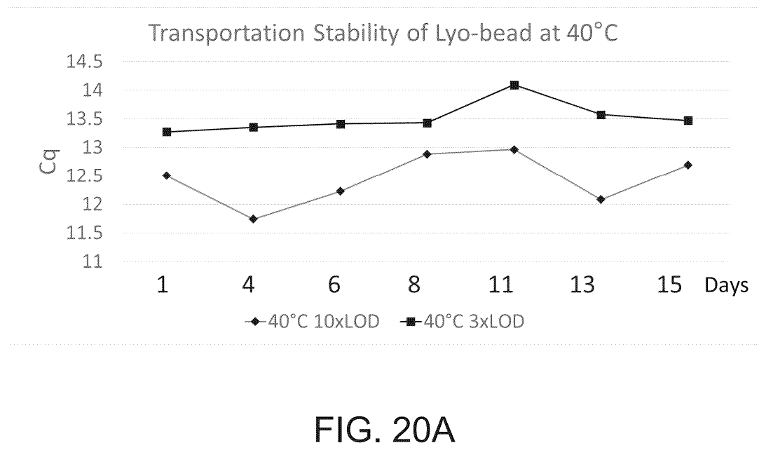

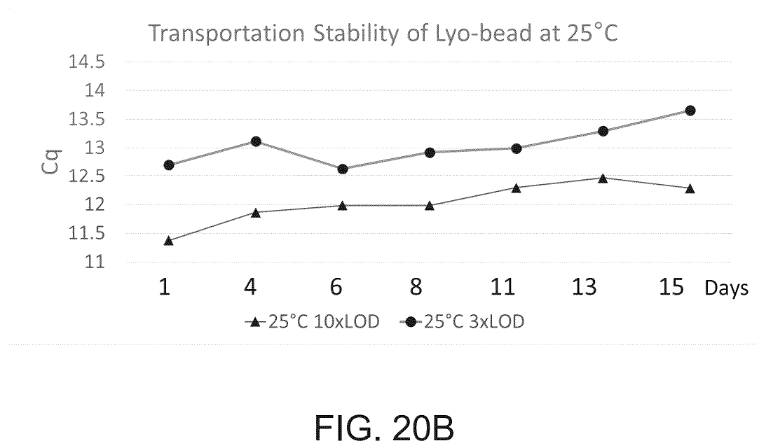

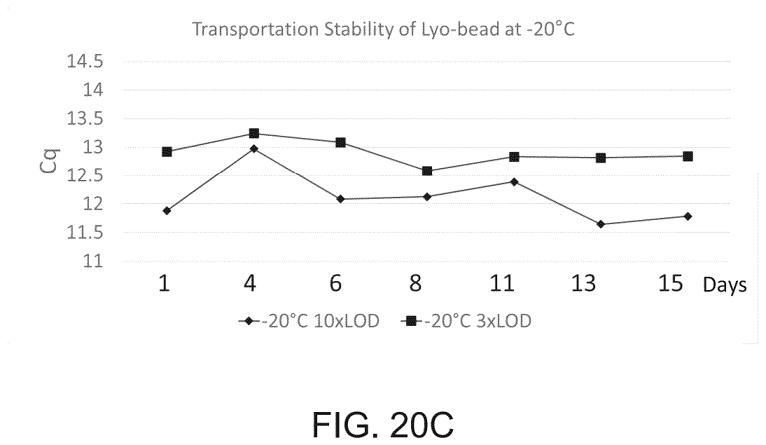

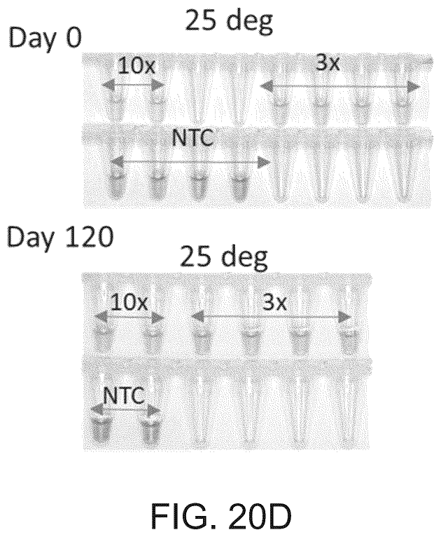

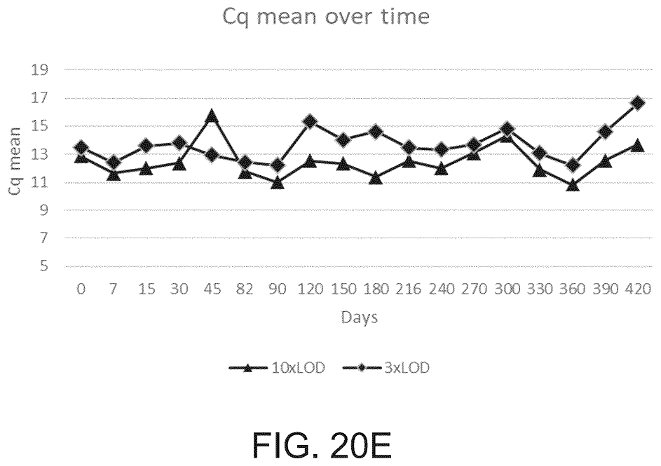

Example 16 illustrates the stability of the lyophilized colorimetric LAMP reaction mixture of the present disclosure. The lyophilized beads were stored in different temperatures across a period of 15 days to investigate the transportation stability while room temperature (25° C.) storage served as real time stability study. FIGS. 20A to 20C show the Cq values detected using 10× and 3× LoD of FluA H1N1 reference materials with the lyophilized beads stored in different temperature across different durations. FIGS. 20D and 20E show the color change and the Cq values detected using 10× and 3× LoD of FluA H1N1 reference materials with the lyophilized beads stored in room temperature (25° C.). The results show that the assay is stable up to 15 days in adverse temperature ranging from 40° C. to −20° C. As for the real time stability study, the assay is stable up to 120 days and the study is still ongoing. Therefore, the present disclosure provides the lyophilized-ready colorimetric LAMP recipe which allows the whole diagnostic assay to be lyophilized into temperature stable colorimetric LAMP reagent without losing the performance for the colorimetric LAMP reaction after rehydration.

From the above, the present disclosure provides the LAMP assay system for detection of respiratory viruses including SARS-CoV-2, FluA and FluB. The LAMP assay system includes the LAMP reaction mixture having the primer kit, and the primer kit includes at least one of the first primer set specific to SARS-CoV-2, the second primer set specific to Influenza A, and the third primer set specific to Influenza B. The three primer sets may be independently used for detecting SARS-CoV-2, FluA and FluB respectively, and may also be combined to be a multiplex detection kit for detecting SARS-CoV-2, FluA and FluB simultaneously. Particularly, additional outer primers are included in the primer sets to improve the speed and sensitivity of the assay. The designed assay of the present disclosure shows better performance when additional outer primers are added.

The present disclosure provides the LAMP assay system for detecting SARS-CoV-2, FluA and FluB with high sensitivity, high specificity, and reduced reaction time. Particularly, the present disclosure provides the recipe for colorimetric LAMP reaction mixture which allows for fast and efficient LAMP amplification and detection with color changes. The assays with color changes have detection limits as low as 1000 copies/ml, 1800 copies/ml and 600 copies/ml for SARS-CoV-2, FluA and FluB, respectively.

The designed assay of the present disclosure can effectively detect all known SARS-CoV-2 (99.8%; n=366707), FluA (H1N1, H3N2, H5N1 and H7N9) and FluB (B/Yamagata and B/Florida) strains. Thus, the present disclosure provides comprehensive detection of all known strains for SARS-CoV-2, FluA and FluB.

The designed assay of the present disclosure provides fast target amplification, which has a turnaround time of 20 min and is faster comparing to other PCR detection system, which takes around 1 hour for RNA detection. The amplification signal in the diagnostic assay of the present disclosure can be detected either with naked eye or any other optical detection system.

The designed assay of the present disclosure can detect the nucleic acid from direct extracted nucleic acid without purification step, and the detection sensitivity is not compromised, suggesting the high inhibitor resistance capability of the assay.

The designed assay of the present disclosure shows no loss of sensitivity after lyophilized, suggesting compatibility with the lyophilized ready colorimetric LAMP and fluorescence LAMP reagents.

The designed assay of the present disclosure enables direct detection of target nucleic acid from real samples (nasal, nasopharyngeal and saliva samples). The diagnostic assay of the present disclosure is able to detect the target directly from real sample with β-actin housekeeping gene as the sample control without prior purification.

While the disclosure has been described in terms of what is presently considered to be the most practical and preferred embodiments, it is to be understood that the disclosure needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A loop-mediated isothermal amplification (LAMP) assay system for detection of a respiratory virus, comprising a LAMP reaction mixture having a primer kit, a strand-displacing polymerase and deoxyribonucleoside triphosphates for amplifying a target sequence,
wherein the primer kit comprises at least one of a first primer set specific to SARS-CoV-2, a second primer set specific to Influenza A, and a third primer set specific to Influenza B,
wherein the first primer set comprises primers having nucleotide sequences of SEQ ID NOs: 1 to 6 and SEQ ID NOs: 13 to 18,
wherein the second primer set comprises primers having nucleotide sequences of SEQ ID NOs: 25 to 30, and
wherein the third primer set comprises primers having nucleotide sequences of SEQ ID NOs: 44 to 49.

2. The LAMP assay system according to claim 1, wherein the first primer set further comprises at least one of primers having nucleotide sequences of SEQ ID NOs: 7 to 12.

3. The LAMP assay system according to claim 1, wherein the first primer set further comprises at least one of primers having nucleotide sequences of SEQ ID NOs: 19 to 24.

4. The LAMP assay system according to claim 1, wherein the second primer set further comprises at least one of primers having nucleotide sequences of SEQ ID NOs: 31 to 37.

5. The LAMP assay system according to claim 1, wherein the second primer set further comprises at least one of primers having nucleotide sequences of SEQ ID NOs: 38 to 43.

6. The LAMP assay system according to claim 1, wherein the third primer set further comprises at least one of primers having nucleotide sequences of SEQ ID NOs: 50 to 59.

7. The LAMP assay system according to claim 1, wherein the primer kit further comprises an internal control primer set specific to β-actin, and the internal control primer set comprises primers having nucleotide sequences of SEQ ID NOs: 60 to 65.

8. The LAMP assay system according to claim 1, wherein the LAMP reaction mixture further comprises a pH indicating dye in a concentration ranged 0.08 to 0.3 mM.

9. The LAMP assay system according to claim 8, wherein the pH indicating dye is phenol red or neutral red.

10. The LAMP assay system according to claim 1, wherein the LAMP reaction mixture further comprises a lyoprotectant sugar in a concentration ranged 1 to 10% (w/v), wherein the lyoprotectant sugar is selected from the group consisting of trehalose, raffinose, dextran, mannitol and mixtures thereof.

11. The LAMP assay system according to claim 10, wherein the LAMP reaction mixture comprises 2 to 7.5% (w/v) of trehalose and 2 to 7.5% (w/v) of raffinose.

12. The LAMP assay system according to claim 10, wherein the LAMP reaction mixture comprises 2 to 7.5% (w/v) of trehalose, 2 to 7.5% (w/v) of raffinose, 1 to 2.5% (w/v) of dextran, and 1 to 5% (w/v) of mannitol.

13. The LAMP assay system according to claim 1, wherein the LAMP reaction mixture further comprises a magnesium salt in a concentration ranged 4 to 12 mM.

14. The LAMP assay system according to claim 1, further comprising an extraction-free lysis buffer, wherein the extraction-free lysis buffer comprises:
potassium chloride in a concentration ranged 10 to 50 mM;
ammonium sulfate in a concentration ranged 10 to 50 mM; and
a detergent in a concentration ranged 0.5 to 6% (w/v), wherein the detergent is selected from the group consisting of 2-ethylhexan-1-ol;2-methyloxirane;oxirane (CAS number: 64366-70-7), secondary alcohol ethoxylate (CAS number: 84133-50-6), 2-[4-(2,4,4-trimethylpentan-2-yl)phenoxy]ethanol (CAS number: 9002-93-1), 2-[4-(2,4,4-trimethylpentan-2-yl)phenoxy]ethan-1-ol (CAS number: 9002-93-1), and {2-[3,4-bis(2-hydroxyethoxy)oxolan-2-yl]-2-(2-hydroxyethoxy)ethoxy}ethyl dodecanoate (CAS number: 9005-64-5) and mixtures thereof.

15. The LAMP assay system according to claim 14, wherein the LAMP reaction mixture is all-in-one lyophilized mixture and is rehydrated with the extraction-free lysis buffer to be ready for nucleic acid amplification and detection.

16. The LAMP assay system according to claim 14, wherein the detergent in the extraction-free lysis buffer is 0.5 to 6% (w/v) of 2-ethylhexan-1-ol;2-methyloxirane;oxirane (CAS number: 64366-70-7).

17. The LAMP assay system according to claim 14, wherein the detergent in the extraction-free lysis buffer is 0.5 to 6% (w/v) of secondary alcohol ethoxylate (CAS number: 84133-50-6).

18. The LAMP assay system according to claim 14, wherein the detergent in the extraction-free lysis buffer is 0.5 to 6% (w/v) of 2-[4-(2,4,4-trimethylpentan-2-yl)phenoxy]ethanol (CAS number: 9002-93-1).

19. The LAMP assay system according to claim 14, wherein the detergent in the extraction-free lysis buffer is 0.5 to 6% (w/v) of 2-[4-(2,4,4-trimethylpentan-2-yl)phenoxy]ethan-1-ol (CAS number: 9002-93-1).

20. The LAMP assay system according to claim 14, wherein the detergent in the extraction-free lysis buffer is 0.5 to 6% (w/v) of {2-[3,4-bis(2-hydroxyethoxy)oxolan-2-yl]-2-(2-hydroxyethoxy)ethoxy}ethyl dodecanoate (CAS number: 9005-64-5).
